(19) United States
(12) Patent Application Publication
(10) Pub. No.: US 2017/0349644 A1
(43) Pub. Date: Dec. 7, 2017

(54) FACTOR VIII WITH EXTENDED HALF-LIFE AND REDUCED LIGAND-BINDING PROPERTIES

(71) Applicants: Baxalta Incorporated, Bannockburn, IL (US); Baxalta GmbH, Glattpark (Opfikon) (CH)

(72) Inventors: Peter Turecek, Klosterneuburg - Weidling (AT); Gerald Schrenk, Vienna (AT); Juergen Siekmann, Vienna (AT)

(21) Appl. No.: 15/369,529

(22) Filed: Dec. 5, 2016

Related U.S. Application Data
(60) Provisional application No. 62/262,674, filed on Dec. 3, 2015.

(51) Int. Cl. C07K 14/755

(52) U.S. Cl. CPC C07K 14/755

(57) ABSTRACT

The invention relates to materials and methods of conjugating a water soluble polymer to an oxidized carbohydrate moiety of a therapeutic protein comprising contacting the oxidized carbohydrate moiety with an activated water soluble polymer under conditions that allow conjugation. More specifically, the present invention relates to a modified, recombinant Factor VIII (FVIII) with extended half-life and reduced ligand-binding properties.

### CROSS-REFERENCE TO RELATED APPLICATIONS

This application claims priority to U.S. Provisional Patent Application No. 62/262,674, filed Dec. 3, 2015, which is hereby incorporated by reference.

### FIELD OF THE INVENTION

The present invention relates to materials and methods for extending the half-life of Factor VIII (FVIII).

### BACKGROUND OF THE INVENTION

Recognized treatment and/or prevention of bleeding in people with hemophilia A and B often includes factor replacement therapy. This involves the infusion (injection into the bloodstream) of blood coagulation proteins such as Factor VIII (FVIII) and Factor IX (FIX). These proteins come typically from two sources: isolation from human plasma and expression in genetically-engineered cell lines. Because the replacement of the missing clotting factors is not permanent, patients receiving such therapy must be repeatedly infused with factor.

The pharmacological and immunological properties of therapeutic proteins such as FVIII can be improved by chemical modification and conjugation with polymeric compounds. Polysialic acid (PSA), also referred to as colominic acid (CA), is a naturally occurring polysaccharide. It is a homopolymer of N-acetylneuraminic acid with α(2→8) ketosidic linkage and contains vicinal diol groups at its non-reducing end. The polymer is negatively charged and is a natural constituent of the human body. It can easily be produced from bacteria in large quantities and with pre-determined physical characteristics (U.S. Pat. No. 5,846,951). Bacterially-produced PSA consists of the same sialic acid monomers as PSA produced in the human body. Unlike some polymers, PSA is biodegradable.

Covalent coupling of colominic acid to catalase and asparaginase has been shown to increase enzyme stability in the presence of proteolytic enzymes or blood plasma. Comparative in vivo studies with polysialylated and unmodified asparaginase revealed that polysialylation increased the half-life of the enzyme (Fernandes and Gregoriadis, Int J Pharm. 2001, 217, 215-24).

There remains a need to develop materials and methods for conjugating water soluble polymers to proteins that improves the protein's pharmacodynamic and/or pharmacokinetic properties while minimizing the costs associated with the various reagents and minimizing the health risks to the patient recipient.

### SUMMARY OF THE INVENTION

The present invention provides materials and methods for conjugating polymers to proteins to improve the protein's in vivo half-life, pharmacodynamic and/or pharmacokinetic properties.

In one embodiment, a modified Factor VIII (FVIII) is provided. The modified FVIII comprises a modification increasing FVIII half-life and reducing binding of the modified FVIII to a ligand binding FVIII. Exemplary ligands are selected from von Willebrand Factor (VWF) and low density lipoprotein (LDL)-receptor-related protein 1 (LRP1). Exemplary modifications are discussed herein and include, for example, chemical modifications such as attachment of water-soluble polymers.

In an exemplary embodiment, the modified FVIII is plasma-derived. In an exemplary embodiment, the FVIII is recombinantly produced from an engineered host cell. In various embodiments, the modified FVIII includes an intact B domain. In various embodiments, the FVIII is a full-length FVIII and includes an intact B domain.

In an exemplary embodiment, the modified FVIII binds to VWF and LRP1 with a lower affinity (KD) compared to an unmodified FVIII.

In an exemplary embodiment, the modified FVIII comprises a polysialic acid (PSA) conjugated thereto. Exemplary PSA moieties of use in this embodiment have a mean molecular weight of about 20 kDa. In exemplary embodiments, the PSA has a mean molecular weight selected from about 5, about 10, about 15, about 20, about 25, about 30, about 35, about 40, or about 50 kDa. In an exemplary embodiment, the PSA has a low polydispersity. Exemplary PSA conjugates include an aminooxy linker between the PSA and the FVIII molecule. An exemplary aminooxy linker is attached to an oxidized carbohydrate of the modified FVIII.

The invention also provides a pharmaceutical composition. An exemplary pharmaceutical formulation includes an aforementioned modified FVIII and a pharmaceutically acceptable carrier, diluent, salt, buffer, and/or excipient.

In various embodiments, an aforementioned modified FVIII has an in vivo half-life that is longer than a PEGylated FVIII. In various embodiments, an aforementioned modified FVIII has an in vivo half-life that is longer than a PEGylated FVIII, which is conjugated to a PEG moiety of about the same mean molecular weight as the mean molecular weight of the PSA of the conjugated FVIII with the longer in vivo half-life. In an exemplary embodiment, the PSA modified FVIII is conjugated to a PSA moiety having a mean molecular weight of about 20 kDa and has an in vivo half-life that is longer than a PEGylated FVIII, which is conjugated to a PEG moiety having a mean molecular weight of about 20 kDa. In various embodiments, the half-life is longer by a factor of about 1-, about 2- or about 3-fold. In still other various embodiments, the half-life is longer by a factor of about 1-, about 2-, about 3-, about 4-, about 5-, about 6-, about 7-, about 8-, about 9-, or about 10-fold.

In an exemplary embodiment, an aforementioned PSA modified FVIII binds to VWF or LRP1 with a lower binding affinity as compared to the binding of a PEGylated FVIII to VWF or LRP1 when the binding of both species is measured under comparable conditions. In an exemplary embodiment, an aforementioned PSA modified FVIII binds to VWF or LRP1 with a lower binding affinity as compared to the binding of a PEGylated FVIII to VWF or LRP1 when the binding of both species is measured under comparable conditions, and the PEG and PSA are of about the same mean molecular weight. In various embodiments, binding to VWF or LRP1 is lower by a factor of about 0.5-fold as compared to binding of a PEGylated FVIII to VWF or LRP1. In still other embodiments, binding to VWF or LRP1 is lower by a factor of about 0.1-, about 0.2-, about 0.3-, about 0.4-, about 0.5-, about 0.6-, about 0.7-, about 0.8-, about 0.-9, about 1-, about 2-, about 3-, about 4-, about 5-, about 6-, about 7-, about 8-, about 9-, or about 10- as compared to binding of a PEGylated FVIII to VWF or LRP1.

In one embodiment, a modified, recombinant FVIII is provided comprising a modification that increases FVIII half-life and reduces binding of said modified FVIII to a ligand selected from the group consisting VWF and LRP1, wherein said modification comprises a PSA attached to a FVIII through an aminooxy linker. In an exemplary embodiment, the aminooxy linker is attached to an oxidized carbohydrate of the modified FVIII, wherein the in vivo half-life of the modified, recombinant FVIII is longer than an unmodified, recombinant FVIII and/or a PEGylated, recombinant FVIII and wherein the binding affinity to VWF or LRP1 of the modified, recombinant FVIII is lower as compared to binding of VWF or LRP1 by an unmodified, recombinant FVIII and/or a PEGylated, recombinant FVIII. In an exemplary embodiment, the comparative species include a PSA and a PEG moiety of about the same mean molecular weight.

The invention further provides a method of treating a subject in need of such treatment with a modified FVIII of the invention. In an exemplary embodiment, a pharmaceutical composition of the invention, including the disclosed modified FVIII is administered to a mammal diagnosed with disease or disorder associated with FVIII deficiency, or deficiency of another factor (e.g., FVII, FIX).

In an exemplary embodiment, the invention provides a method of treating a hemorrhagic defect in a mammalian subject. An exemplary method includes administering an aforementioned modified FVIII or a pharmaceutical composition thereof to the subject, in need of such treatment, in an amount effective to reduce or eliminate one or more symptoms of the hemorrhagic defect. In an exemplary embodiment, the administration of the pharmaceutical formulation achieves an amount effective to reduce or eliminate one or more symptoms of the hemorrhagic defect when administered to the subject not more than once every 4 days, not more than once every 5 days, not more than once every 6 days, not more than once every 7 days, not more than once every 8 days, not more than once every 9 days, or not more than once every 10 days.

### FIGURES

FIG. 1 shows binding signals expressed as Rₘₐₓ, which is the calculated maximum binding at saturation, for PSA-rFVIII groups and rebuffered rFVIII at the three different densities of the sensor-chip-immobilized VWF.

FIG. 2 shows results for FVIII protein-concentration-dependent binding to LRP1 for PSA-rFVIII and for re-buffered rFVIII.

FIG. 3 shows that rFVIII binds strongly to LRP1, PEG-rFVIII displayed residual association with LRP1, and PEG-rFVIII was virtually unable to associate with LRP1.

FIG. 4 shows FXa generation over time after activation of FVIII by thrombin.

FIG. 5 shows the results of the evaluation of the peak thrombin generation curves for rebuffered rFVIII and PSA-rFVIII.

FIG. 6 shows total thrombin generation curves for rebuffered rFVIII and PSA-rFVIII.

FIG. 7 shows PEGylated and polysialylated rFVIII showed improved PK parameters compared to rFVIII in mice.

FIG. 8 shows PEGylated and polysialylated rFVIII showed improved PK parameters compared to rFVIII in rats.

FIG. 9 shows PEGylated and polysialylated rFVIII showed improved PK parameters compared to rFVIII in macaques.

### DETAILED DESCRIPTION OF THE INVENTION

The pharmacological and immunological properties of therapeutic proteins can be improved by chemical modification and conjugation with polymeric compounds such as polysialic acid (PSA). The properties of the resulting conjugates generally strongly depend on the structure and the size of the polymer. Thus, polymers with a defined and narrow size distribution are usually preferred in the art. Synthetic polymers like PEG can be manufactured easily with a narrow size distribution, while PSA can be purified in such a manner that results in a final PSA preparation with a narrow size distribution.

As described herein, the addition of a soluble polymer, such as through polysialylation, is one approach to improve the properties of therapeutic proteins such as FVIII.

### Therapeutic Proteins

### Blood Coagulation Proteins

In one aspect, the starting material of the present invention is a blood coagulation protein, which can be derived from human plasma, or produced by recombinant engineering techniques, as described in U.S. Pat. No. 4,757,006; U.S. Pat. No. 5,733,873; U.S. Pat. No. 5,198,349; U.S. Pat. No. 5,250,421; U.S. Pat. No. 5,919,766; and EP 306 968. Additional recent examples include U.S. Pat. No. 7,645,860; U.S. Pat. No. 8,637,640; U.S. Pat. No. 8,642,737; and U.S. Pat. No. 8,809,501.

Therapeutic polypeptides such as blood coagulation proteins including Factor IX (FIX), Factor VIII (FVIII), Factor VIIa (FVIIa), Von Willebrand Factor (VWF), Factor FV (FV), Factor X (FX), Factor XI (FXI), Factor XII (FXII), thrombin (FII), protein C, protein S, tPA, PAI-1, tissue factor (TF) and ADAMTS 13 protease are rapidly degraded by proteolytic enzymes and neutralized by antibodies. This reduces their half-life and circulation time, thereby limiting their therapeutic effectiveness. Relatively high doses and frequent administration are necessary to reach and sustain the desired therapeutic or prophylactic effect of these coagulation proteins. As a consequence, adequate dose regulation is difficult to obtain and the need of frequent intravenous administrations imposes restrictions on the patient's way of living.

As described herein, blood coagulation proteins including, but not limited to, Factor IX (FIX), Factor VIII (FVIII), Factor VIIa (FVIIa), Von Willebrand Factor (VWF), Factor FV (FV), Factor X (FX), Factor XI, Factor XII (FXII), thrombin (FII), protein C, protein S, tPA, PAI-1, tissue factor (TF) and ADAMTS 13 protease are contemplated by the invention. As used herein, the term “blood coagulation protein” refers to any Factor IX (FIX), Factor VIII (FVIII), Factor VIIa (FVIIa), Von Willebrand Factor (VWF), Factor FV (FV), Factor X (FX), Factor XII (FXII), thrombin (FII), protein C, protein S, tPA, PAI-1, tissue factor (TF) and ADAMTS 13 protease which exhibits biological activity that is associated with that particular native blood coagulation protein.

The blood coagulation cascade is divided into three distinct segments: the intrinsic, extrinsic, and common pathways (Schenone et al., Curr Opin Hematol. 2004, 11, 272-7). The cascade involves a series of serine protease enzymes (zymogens) and protein cofactors. When required, an inactive zymogen precursor is converted into the active form, which consequently converts the next enzyme in the cascade.

The intrinsic pathway requires the clotting factors VIII, IX, X, XI, and XII. Initiation of the intrinsic pathway occurs when prekallikrein, high-molecular-weight kininogen, factor XI (FXI) and factor XII (FXII) are exposed to a negatively charged surface. Also required are calcium ions and phospholipids secreted from platelets.

The extrinsic pathway is initiated when the vascular lumen of blood vessels is damaged. The membrane glycoprotein tissue factor is exposed and then binds to circulating factor VII (FVII) and to small preexisting amounts of its activated form FVIIa. This binding facilitates full conversion of FVII to FVIIa and subsequently, in the presence of calcium and phospholipids, the conversion of factor IX (FIX) to factor IXa (FIXa) and factor X (FX) to factor Xa (FXa). The association of FVIIa with tissue factor enhances the proteolytic activity by bringing the binding sites of FVII for the substrate (FIX and FX) into closer proximity and by inducing a conformational change, which enhances the enzymatic activity of FVIIa.

The activation of FX is the common point of the two pathways. Along with phospholipid and calcium, factors Va (FVa) and Xa convert prothrombin to thrombin (prothrombinase complex), which then cleaves fibrinogen to form fibrin monomers. The monomers polymerize to form fibrin strands. Factor XIIIa (FXIIIa) covalently bonds these strands to one another to form a rigid mesh.

Conversion of FVII to FVIIa is also catalyzed by a number of proteases, including thrombin, FIXa, FXa, factor XIa (FXIa), and factor XIIa (FXIIa). For inhibition of the early phase of the cascade, tissue factor pathway inhibitor targets FVIIa/tissue factor/FXa product complex.

### Factor VIII

Coagulation factor VIII (FVIII) circulates in plasma at a very low concentration and is bound non-covalently to Von Willebrand factor (VWF). During hemostasis, FVIII is separated from VWF and acts as a cofactor for activated factor IX (FIXa)-mediated FX activation by enhancing the rate of activation in the presence of calcium and phospholipids or cellular membranes.

FVIII activated by thrombin has been implicated in binding to Low Density Lipoprotein Receptor Protein (hereinafter referred to as “LRP”) (Yakhyaev, A. et al., Blood, vol. 90 (Suppl. 1), 1997, 126-I, incorporated herein by reference). It has also been demonstrated that non-activated FVIII interacts with the multifunctional endocytic receptor low-density lipoprotein receptor-related protein (LRP) (Lenting, P. J., Neels, J. G., van den Berg, B. M. M., Clijsters, P. P. F. M., Meijerman, D. W. E., Pannekoek, H., van Mourik, J. A., Mertens, K., and van Zonneveld, A.,-J. J. Biol. Chem. 1999, 274, 23734-23739; WO 00/28021; Saenko, E. L., Yakhyaev, A. V., Mikhailenko, I., Strickland, D. K., and Sarafanov, A. G. J. Biol. Chem. 1999, 274, 37685-37692; incorporated herein by reference). It is suggested that this receptor plays a role in the clearance of FVIII from the circulation (Saenko, E. L., et al, supra; Schwarz, H. P., Lenting, P. J., Binder, B., Mihaly, J., Denis, C., Domer, F., and Turecek, P. L. Blood 2000, 95, 1703-1708; incorporated herein by reference).

LRP is a member of the low-density lipoprotein (LDL) receptor family that also includes LDL receptor, very low-density lipoprotein receptor, a polipoprotein E receptor 2, and megalin (for reviews see Neels J. G., Horn, I. R., van den Berg, B. M. M., Pannekoek, H., and van Zonneveld, A.-J. Fibrinolysis Proteolysis 1998, 12, 219-240; Herz, J., and Strickland, D. K. J. Clin. Invest. 2001, 108, 779-784; incorporated herein by reference). It is expressed in a variety of tissues, including liver, lung, placenta, and brain (Moestrup, S. K., Gliemann, J., and Pallesen, G. Cell Tissue Res. 1992, 269, 375-382; incorporated herein by reference). The receptor consists of an extracellular 515-kDa alpha chain, which is non-covalently linked to a transmembrane 85-kDa.beta.-chain (Herz, J., Kowal, R. C., Goldstein, J. L., and Brown, M. S. EMBO J. 1990, 9, 1769-1776; incorporated herein by reference). The alpha-chain contains four clusters of a varying number of complement-type repeats that mediate the binding of many structurally and functionally unrelated ligands (Moestrup, S. K., Hotlet, T. L., Etzerodt, M., Thogersen, H. C., Nykjaer, A., Andreasen, P. A., Rasmussen, H. H., Sottrup-Jensen, L., and Gliemann, J. J. Biol. Chem. 1993, 268, 13691-13696; Willnow, T. E., Orth, K., and Herz, J. J. Biol. Chem. 1994, 269, 15827-15832; Neels, J. G., van den Berg, B. M. M., Lookene, A., Olivecrona, G., Pannekoek, H., and van Zonneveld, A.-J. J. Biol. Chem. 1999, 274, 31305-31311; incorporated herein by reference).

The beta-chain comprises a trans-membrane domain and a short cytoplasmatic tail which is essential for endocytosis. The alpha-chain functions as a large ectodomain and comprises three types of repeats: epidermic growth-Factor-like domains, Tyr-Trp-Thr-Asp sequences and LDL-receptor-class A domains. These class A domains, which have been implicated in ligand binding, are present in four separate clusters which are called Cluster I (2 domains), Cluster II (8 domains), Cluster III (10 domains) and Cluster IV (11 domains).

LRP also binds the activated, non-enzymatic cofactor Factor Villa (Yakhyaev, A. et al., Blood, vol. 90, (Suppl. 1), 1997, 126-I). FVIII light chain has been demonstrated to interact with recombinant LRP clusters II and IV, whereas no binding was observed to LRP clusters I and III (Neels, J. G., et al 1999 supra).

FVIII is synthesized as a single-chain precursor of approximately 270-330 kD with the domain structure A1-A2-B-A3-C1-C2. When purified from plasma (e.g., “plasma-derived” or “plasmatic”), FVIII is composed of a heavy chain (A1-A2-B) and a light chain (A3-C1-C2). The molecular mass of the light chain is 80 kD whereas, due to proteolysis within the B domain, the heavy chain is in the range of 90-220 kD.

FVIII is also synthesized as a recombinant protein for therapeutic use in bleeding disorders. Various in vitro assays have been devised to determine the potential efficacy of recombinant FVIII (rFVIII) as a therapeutic medicine. These assays mimic the in vivo effects of endogenous FVIII. In vitro thrombin treatment of FVIII results in a rapid increase and subsequent decrease in its procoagulant activity, as measured by in vitro assays. This activation and inactivation coincides with specific limited proteolysis both in the heavy and the light chains, which alter the availability of different binding epitopes in FVIII, e.g. allowing FVIII to dissociate from VWF and bind to a phospholipid surface or altering the binding ability to certain monoclonal antibodies.

The lack or dysfunction of FVIII is associated with the most frequent bleeding disorder, hemophilia A. The treatment of choice for the management of hemophilia A is replacement therapy with plasma derived or rFVIII concentrates. Patients with severe hemophilia A with FVIII levels below 1%, are generally on prophylactic therapy with the aim of keeping FVIII above 1% between doses. Taking into account the average half-lives of the various FVIII products in the circulation, this result can usually be achieved by giving FVIII two to three times a week.

Reference polynucleotide and polypeptide sequences include, e.g., UniProtKB/Swiss-Prot P00451 (FA8_HUMAN); Gitschier J et al., Characterization of the human Factor VIII gene, Nature, 1984, 312(5992), 326-30; Vehar G H et al., Structure of human Factor VIII, Nature, 1984, 312(5992), 337-42; Thompson A R. Structure and Function of the Factor VIII gene and protein, Semin Thromb Hemost, 2002, 2003: 29, 11-29.

### Polypeptides

In one aspect, the starting material of the present invention is a protein or polypeptide. As described herein, the term therapeutic protein refers to any therapeutic protein molecule which exhibits biological activity that is associated with the therapeutic protein. In one embodiment of the invention, the therapeutic protein molecule is a full-length protein. In one embodiment, the therapeutic protein is FVIII that has been modified to prolong half-life.

Therapeutic protein molecules contemplated include full-length proteins, precursors of full length proteins, biologically active subunits or fragments of full length proteins, as well as biologically active derivatives and variants of any of these forms of therapeutic proteins. Thus, therapeutic protein include those that (1) have an amino acid sequence that has greater than about 60%, about 65%, about 70%, about 75%, about 80%, about 85%, about 90%, about 91%, about 92%, about 93%, about 94%, about 95%, about 96%, about 97%, about 98% or about 99% or greater amino acid sequence identity, over a region of at least about 25, about 50, about 100, about 200, about 300, about 400, or more amino acids, to a polypeptide encoded by a referenced nucleic acid or an amino acid sequence described herein; and/or (2) specifically bind to antibodies, e.g., polyclonal or monoclonal antibodies, generated against an immunogen comprising a referenced amino acid sequence as described herein, an immunogenic fragment thereof, and/or a conservatively modified variant thereof.

According to the present invention, the term “recombinant therapeutic protein” includes any therapeutic protein obtained via recombinant DNA technology. In certain embodiments, the term encompasses proteins as described herein.

As used herein, “endogenous therapeutic protein” includes a therapeutic protein which originates from the mammal intended to receive treatment. The term also includes therapeutic protein transcribed from a transgene or any other foreign DNA present in said mammal. As used herein, “exogenous therapeutic protein” includes a blood coagulation protein which does not originate from the mammal intended to receive treatment.

As used herein, “plasma-derived,” “plasma-derived blood coagulation protein” or “plasmatic” includes all forms of the protein found in blood obtained from a mammal having the property participating in the coagulation pathway.

As used herein “biologically active derivative” or “biologically active variant” includes any derivative or variant of a molecule having substantially the same functional and/or biological properties of said molecule, such as binding properties, and/or the same structural basis, such as a peptidic backbone or a basic polymeric unit.

An “analog,” such as a “variant” or a “derivative,” is a compound substantially similar in structure and having the same biological activity, albeit in certain instances to a differing degree, to a naturally-occurring molecule. For example, a polypeptide variant refers to a polypeptide sharing substantially similar structure and having the same biological activity as a reference polypeptide. Variants or analogs differ in the composition of their amino acid sequences compared to the naturally-occurring polypeptide from which the analog is derived, based on one or more mutations involving (i) deletion of one or more amino acid residues at one or more termini of the polypeptide and/or one or more internal regions of the naturally-occurring polypeptide sequence (e.g., fragments), (ii) insertion or addition of one or more amino acids at one or more termini (typically an “addition” or “fusion”) of the polypeptide and/or one or more internal regions (typically an “insertion”) of the naturally-occurring polypeptide sequence or (iii) substitution of one or more amino acids for other amino acids in the naturally-occurring polypeptide sequence. By way of example, a “derivative” is a type of analog and refers to a polypeptide sharing the same or substantially similar structure as a reference polypeptide that has been modified, e.g., chemically.

A variant polypeptide is a type of analog polypeptide and includes insertion variants, wherein one or more amino acid residues are added to a therapeutic protein amino acid sequence of the invention. Insertions may be located at either or both termini of the protein, and/or may be positioned within internal regions of the therapeutic protein amino acid sequence. Insertion variants, with additional residues at either or both termini, include for example, fusion proteins and proteins including amino acid tags or other amino acid labels. In one aspect, the blood coagulation protein molecule optionally contains an N-terminal Met, especially when the molecule is expressed recombinantly in a bacterial cell such as E. coli.

In deletion variants, one or more amino acid residues in a therapeutic protein polypeptide as described herein are removed. Deletions can be effected at one or both termini of the therapeutic protein polypeptide, and/or with removal of one or more residues within the therapeutic protein amino acid sequence. Deletion variants, therefore, include fragments of a therapeutic protein polypeptide sequence.

In substitution variants, one or more amino acid residues of a therapeutic protein polypeptide are removed and replaced with alternative residues. In one aspect, the substitutions are conservative in nature and conservative substitutions of this type are well known in the art. Alternatively, the invention embraces substitutions that are also non-conservative. Exemplary conservative substitutions are described in Lehninger, [Biochemistry, 2nd Edition; Worth Publishers, Inc., New York (1975), pp. 71-77] and are set out immediately below.

CONSERVATIVE SUBSTITUTIONS

| SIDE CHAIN | |
|---|---|
| CHARACTERISTIC | AMINO ACID |
| Non-polar (hydrophobic): | |
| A. Aliphatic | A L I V P |
| B. Aromatic | F W |
| C. Sulfur-containing | M |
| D. Borderline | G |
| Uncharged-polar: | |
| A. Hydroxyl | S T Y |
| B. Amides | N Q |
| C. Sulfhydryl | C |
| D. Borderline | G |
| Positively charged (basic) | K R H |
| Negatively charged (acidic) | D E |

Alternatively, exemplary conservative substitutions are set out immediately below.

CONSERVATIVE SUBSTITUTIONS II

| | EXEMPLARY |
|---|---|
| ORIGINAL RESIDUE | SUBSTITUTION |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met (M) | Leu, Phe, Ile |
| Phe (F) | Leu, Val, Ile, Ala |
| Pro (P) | Gly |
| Ser (S) | Thr |
| Thr (T) | Ser |
| Trp (W) | Tyr |
| Tyr (Y) | Trp, Phe, Thr, Ser |
| Val (V) | Ile, Leu, Met, Phe, Ala |

### Polynucleotides

Nucleic acids encoding a therapeutic protein of the invention include, for example and without limitation, genes, pre-mRNAs, mRNAs, cDNAs, polymorphic variants, alleles, synthetic and naturally-occurring mutants.

Polynucleotides encoding a therapeutic protein of the invention also include, without limitation, those that (1) specifically hybridize under stringent hybridization conditions to a nucleic acid encoding a referenced amino acid sequence as described herein, and conservatively modified variants thereof; (2) have a nucleic acid sequence that has greater than about 95%, about 96%, about 97%, about 98%, about 99%, or higher nucleotide sequence identity, over a region of at least about 25, about 50, about 100, about 150, about 200, about 250, about 500, about 1000, or more nucleotides (up to the full length sequence of 1218 nucleotides of the mature protein), to a reference nucleic acid sequence as described herein. Exemplary “stringent hybridization” conditions include hybridization at 42° C. in 50% formamide, 5×SSC, 20 mM Na.PO4, pH 6.8; and washing in 1×SSC at 55° C. for 30 minutes. It is understood that variation in these exemplary conditions can be made based on the length and GC nucleotide content of the sequences to be hybridized. Formulas standard in the art are appropriate for determining appropriate hybridization conditions. See Sambrook et al., Molecular Cloning: A Laboratory Manual (Second ed., Cold Spring Harbor Laboratory Press, 1989) §§9.47-9.51.

A “naturally-occurring” polynucleotide or polypeptide sequence is typically derived from a mammal including, but not limited to, primate, e.g., human; rodent, e.g., rat, mouse, hamster; cow, pig, horse, sheep, or any mammal. The nucleic acids and proteins of the invention can be recombinant molecules (e.g., heterologous and encoding the wild type sequence or a variant thereof, or non-naturally occurring).

### Production of Therapeutic Proteins

Production of a therapeutic protein includes any method known in the art for (i) the production of recombinant DNA by genetic engineering, (ii) introducing recombinant DNA into prokaryotic or eukaryotic cells by, for example and without limitation, transfection, electroporation or microinjection, (iii) cultivating said transformed cells, (iv) expressing therapeutic protein, e.g. constitutively or upon induction, and (v) isolating said blood coagulation protein, e.g. from the culture medium or by harvesting the transformed cells, in order to obtain purified therapeutic protein.

In other aspects, the therapeutic protein is produced by expression in a suitable prokaryotic or eukaryotic host system characterized by producing a pharmacologically acceptable blood coagulation protein molecule. Examples of eukaryotic cells are mammalian cells, such as CHO, COS, HEK 293, BHK, SK-Hep, and HepG2.

A wide variety of vectors are used for the preparation of the therapeutic protein and are selected from eukaryotic and prokaryotic expression vectors. Examples of vectors for prokaryotic expression include plasmids such as, and without limitation, pRSET, pET, and pBAD, wherein the promoters used in prokaryotic expression vectors include one or more of, and without limitation, lac, trc, trp, recA, or araBAD. Examples of vectors for eukaryotic expression include: (i) for expression in yeast, vectors such as, and without limitation, pAO, pPIC, pYES, or pMET, using promoters such as, and without limitation, AOX1, GAP, GAL1, or AUG1; (ii) for expression in insect cells, vectors such as and without limitation, pMT, pAc5, pIB, pMIB, or pBAC, using promoters such as and without limitation PH, p10, MT, Ac5, OpIE2, gp64, or polh, and (iii) for expression in mammalian cells, vectors such as and without limitation pSVL, pCMV, pRc/RSV, pcDNA3, or pBPV, and vectors derived from, in one aspect, viral systems such as and without limitation vaccinia virus, adeno-associated viruses, herpes viruses, or retroviruses, using promoters such as and without limitation CMV, SV40, EF-1, UbC, RSV, ADV, BPV, and β-actin.

Additional recent examples include U.S. Pat. No. 7,645,860; U.S. Pat. No. 8,637,640; U.S. Pat. No. 8,642,737; and U.S. Pat. No. 8,809,501.

### Administration

In one embodiment a conjugated therapeutic protein of the present invention may be administered by injection, such as intravenous, intramuscular, or intraperitoneal injection.

To administer compositions comprising a conjugated therapeutic protein of the present invention to human or test animals, in one aspect, the compositions comprise one or more pharmaceutically acceptable carriers. The terms “pharmaceutically” or “pharmacologically acceptable” refer to molecular entities and compositions that are stable, inhibit protein degradation such as aggregation and cleavage products, and in addition do not produce allergic, or other adverse reactions when administered using routes well-known in the art, as described below. “Pharmaceutically acceptable carriers” include any and all clinically useful solvents, dispersion media, coatings, antibacterial and antifungal agents, isotonic and absorption delaying agents and the like, including those agents disclosed above.

As used herein, “effective amount” includes a dose suitable for treating a disease or disorder or ameliorating a symptom of a disease or disorder. In one embodiment, “effective amount” includes a dose suitable for treating a mammal having a bleeding disorder as described herein.

The compositions may be administered orally, topically, transdermally, parenterally, by inhalation spray, vaginally, rectally, or by intracranial injection. The term parenteral as used herein includes subcutaneous injections, intravenous, intramuscular, intracisternal injection, or infusion techniques. Administration by intravenous, intradermal, intramuscular, intramammary, intraperitoneal, intrathecal, retrobulbar, intrapulmonary injection and or surgical implantation at a particular site is contemplated as well. Generally, compositions are essentially free of pyrogens, as well as other impurities that could be harmful to the recipient.

Single or multiple administrations of the compositions can be carried out with the dose levels and pattern being selected by the treating physician. For the prevention or treatment of disease, the appropriate dosage will depend on the type of disease to be treated, as described above, the severity and course of the disease, whether drug is administered for preventive or therapeutic purposes, previous therapy, the patient's clinical history and response to the drug, and the discretion of the attending physician.

The present invention also relates to a pharmaceutical composition comprising an effective amount of a conjugated therapeutic protein as defined herein. The pharmaceutical composition may further comprise a pharmaceutically acceptable carrier, diluent, salt, buffer, or excipient. The pharmaceutical composition can be used for treating the above-defined bleeding disorders. The pharmaceutical composition of the invention may be a solution or a lyophilized product. Solutions of the pharmaceutical composition may be subjected to any suitable lyophilization process.

As an additional aspect, the invention includes kits which comprise a composition of the invention packaged in a manner which facilitates its use for administration to subjects. In one embodiment, such a kit includes a compound or composition described herein (e.g., a composition comprising a conjugated therapeutic protein), packaged in a container such as a sealed bottle or vessel, with a label affixed to the container or included in the package that describes use of the compound or composition in practicing the method. In one embodiment, the kit contains a first container having a composition comprising a conjugated therapeutic protein and a second container having a physiologically acceptable reconstitution solution for the composition in the first container. In one aspect, the compound or composition is packaged in a unit dosage form. The kit may further include a device suitable for administering the composition according to a specific route of administration. Preferably, the kit contains a label that describes use of the therapeutic protein or peptide composition.

### Water Soluble Polymers

In one aspect, a therapeutic protein derivative (i.e., a conjugated therapeutic protein) molecule provided is bound to a water-soluble polymer including, but not limited to, polyethylene glycol (PEG), branched PEG, polysialic acid (PSA), hydroxyalkyl starch (HAS), hydroxylethyl starch (HES), carbohydrate, polysaccharides, pullulane, chitosan, hyaluronic acid, chondroitin sulfate, dermatan sulfate, starch, dextran, carboxymethyl-dextran, polyalkylene oxide (PAO), polyalkylene glycol (PAG), polypropylene glycol (PPG) polyoxazoline, poly acryloylmorpholine, polyvinyl alcohol (PVA), polycarboxylate, polyvinylpyrrolidone, polyphosphazene, polyoxazoline, polyethylene-co-maleic acid anhydride, polystyrene-co-maleic acid anhydride, poly(l-hydroxymethylethylene hydroxymethylformal) (PHF), 2-methacryloyloxy-2′-ethyltrimethylammoniumphosphate (MPC). In one embodiment of the invention, the water soluble polymer is consisting of sialic acid molecule having a molecular weight range of 350 to 120,000 Da, 500 to 100,000 Da, 1000 to 80,000 Da, 1500 to 60,000 Da, 2,000 to 45,000 Da, 3,000 to 35,000 Da, and 5,000 to 25,000 Da. The coupling of the water soluble polymer can be carried out by direct coupling to the protein or via linker molecules. One example of a chemical linker is MBPH (4-[4-N-Maleimidophenyl]butyric acid hydrazide) containing a carbohydrate-selective hydrazide and a sulfhydryl-reactive maleimide group (Chamow et al., J Biol Chem 1992, 267, 15916-22). Other exemplary and preferred linkers are described below.

### Homobifunctional Linkers

In an exemplary embodiment, the coupling of the water soluble polymer is carried out via homobifunctional linker. In an exemplary embodiment, the homobifunctional linker possess identical reactive groups at opposite ends of the crosslinker's spacer arm and has the formula Y-L-Y. In an exemplary embodiment, the homobifunctional linker is NH₂[OCH₂CH₂]ₙ.ONH₂ where n′=1-10. In an exemplary embodiment, the homobifunctional linker is NH₂[OCH₂CH₂]₂ONH₂. In an exemplary embodiment, the homobifunctional linker is NH₂[OCH₂CH₂]₄ONH₂. In an exemplary embodiment, the homobifunctional linker is NH₂[OCH₂CH₂]₆ONH₂. In an exemplary embodiment, the homobifunctional linker is NH₂[OCH₂CH₂]₈ONH₂. In an exemplary embodiment, the homobifunctional linker is NH₂[OCH₂CH₂]₁₀ONH₂.

In one embodiment, the derivative retains the full functional activity of native therapeutic protein products, and provides an extended half-life in vivo, as compared to native therapeutic protein products. In an exemplary embodiment, the derivative retains at least about 20, about 21, about 22, about 23, about 24, about 25, about 26, about 27, about 28, about 29, about 30, about 31, about 32, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, 110, about 120, about 130, about 140, or about 150 percent (%) biological activity relative to native blood coagulation protein. In an exemplary embodiment, the PSA-rFVIII conjugate obtains a specific activity of about 70% greater relative to native rFVIII. In an exemplary embodiment, the biological activities of the derivative and native blood coagulation protein are determined by the ratios of chromogenic activity to blood coagulation factor antigen value (blood coagulation factor:Chr: blood coagulation factor:Ag). In an exemplary embodiment of the invention, the half-life of the construct is decreased or increased about 1.0, about 1.1, about 1.2, about 1.3, about 1.4, about 1.5, about 2, about 3, about 4, about 5, about 6, about 7, about 8, about 9, or about 10-fold relative to the in vivo half-life of native therapeutic protein.

In an exemplary embodiment, the derivative, e.g., a modified FVIII that includes a PSA as described herein, is modified in such a way so as to reduce or otherwise limit the ability of the modified FVIII to interact (e.g., bind) to one or more ligands such as VWF or LRP1. For example, a modified FVIII according to the instant disclosure includes a FVIII upon which 1, 2, 3, 4, 5, 6, 7, 8, 9, 10, 11, 12, 13, 14, 15 or more PSA moieties are attached either directly to separate amino acids of FVIII or to, for example, carbohydrate moieties on the FVIII. As disclosed herein, an aminooxy linker is contemplated where attachment occurs via a FVIII carbohydrate moiety.

In various embodiments, the binding affinity of the modified FVIII to, for example VWF and/or LRP1, directly correlates with the half-life of the modofoed FVIII. For example, the stronger the binding to VWF, the more the half life is controlled by VWF and not by, for example, the water-soluble polymer. In this way, a modification that more severely reduces VWF binding will control half-life prolongation on the basis of the clearance-deterring properties conferred by the water-soluble polymer as opposed to the VWF.

### Sialic Acid and PSA

PSAs consist of polymers (generally homopolymers) of N-acetylneuraminic acid. The secondary amino group normally bears an acetyl group, but it may instead bear a glycolyl group. Possible substituents on the hydroxyl groups include acetyl, lactyl, ethyl, sulfate, and phosphate groups.

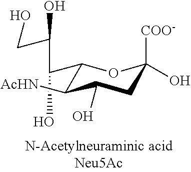

### Structure of Sialic Acid (N-acetylneuraminic Acid)

PSAs and mPSAs generally comprise linear polymers consisting essentially of N-acetylneuraminic acid moieties linked by 2,8- or 2,9-glycosidic linkages or combinations of these (e.g. alternating 2,8- and 2,9-linkages). In particularly preferred PSAs and mPSAs, the glycosidic linkages are α-2,8. Such PSAs and mPSAs are conveniently derived from colominic acids, and are referred to herein as “CAs” and “mCAs”. Typical PSAs and mPSAs comprise at least 2, preferably at least 5, more preferably at least 10 and most preferably at least 20 N-acetylneuraminic acid moieties. Thus, they may comprise from 2 to 300 N-acetylneuraminic acid moieties, preferably from 5 to 200 N-acetylneuraminic acid moieties, or most preferably from 10 to 100 N-acetylneuraminic acid moieties. PSAs and CAs preferably are essentially free of sugar moieties other than N-acetylneuraminic acid. Thus PSAs and CAs preferably comprise at least 90%, more preferably at least 95% and most preferably at least 98% N-acetylneuraminic acid moieties.

### Oxidation of Moieties

Where PSAs and CAs comprise moieties other than N-acetylneuraminic acid (as, for example in mPSAS and mCAs) these are preferably located at one or both of the ends of the polymer chain. Such “other” moieties may, for example, be moieties derived from terminal N-acetylneuraminic acid moieties by oxidation or reduction.

For example, WO-A-0187922 describes such mPSAs and mCAs in which the non-reducing terminal N-acetylneuraminic acid unit is converted to an aldehyde group by reaction with sodium periodate. Additionally, WO 2005/016974 describes such mPSAs and mCAs in which the reducing terminal N-acetylneuraminic acid unit is subjected to reduction to reductively open the ring at the reducing terminal N-acetylneuraminic acid unit, whereby a vicinal diol group is formed, followed by oxidation to convert the vicinal diol group to an aldehyde group.

Sialic acid rich glycoproteins bind selectin in humans and other organisms. They play an important role in human influenza infections. E.g., sialic acid can hide mannose antigens on the surface of host cells or bacteria from mannose-binding lectin. This prevents activation of complement. Sialic acids also hide the penultimate galactose residue thus preventing rapid clearance of the glycoprotein by the galactose receptor on the hepatic parenchymal cells.

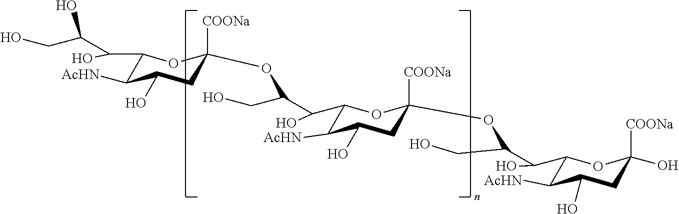

Structure of Colominic Acid (Homopolymer of N-acetylneuraminic Acid)

Colominic acids (a sub-class of PSAs) are homopolymers of N-acetylneuraminic acid (NANA) with α (2→8) ketosidic linkage, and are produced, inter alia, by particular strains of Escherichia coli possessing K1 antigen. Colominic acids have many physiological functions. They are important as a raw material for drugs and cosmetics.

Comparative studies in vivo with polysialylated and unmodified asparaginase revealed that polysialylation increased the half-life of the enzyme (Fernandes and Gregoriadis, Biochimica Biophysica Acta 1997, 1341, 26-34).

As used herein, “sialic acid moieties” includes sialic acid monomers or polymers (“polysaccharides”) which are soluble in an aqueous solution or suspension and have little or no negative impact, such as side effects, to mammals upon administration of the PSA-blood coagulation protein conjugate in a pharmaceutically effective amount. The polymers are characterized, in one aspect, as having about 1, about 2, about 3, about 4, about 5, about 10, about 20, about 30, about 40, about 50, about 60, about 70, about 80, about 90, about 100, about 200, about 300, about 400, or about 500 sialic acid units. In certain aspects, different sialic acid units are combined in a chain.

In one embodiment of the invention, the sialic acid portion of the polysaccharide compound is highly hydrophilic, and in an exemplary embodiment the entire compound is highly hydrophilic. Hydrophilicity is conferred primarily by the pendant carboxyl groups of the sialic acid units, as well as the hydroxyl groups. The saccharide unit may contain other functional groups, such as, amine, hydroxyl or sulphate groups, or combinations thereof. These groups may be present on naturally-occurring saccharide compounds, or introduced into derivative polysaccharide compounds.

The naturally occurring polymer PSA is available as a polydisperse preparation showing a broad size distribution (e.g. Sigma C-5762) and high polydispersity (PD). Because the polysaccharides are usually produced in bacteria carrying the inherent risk of copurifying endotoxins, the purification of long sialic acid polymer chains may raise the probability of increased endotoxin content. Short PSA molecules with 1-4 sialic acid units can also be synthetically prepared (Kang S H et al., Chem Commun. 2000; 227-8; Ress D K and Linhardt R J, Current Organic Synthesis. 2004; 1:31-46), thus minimizing the risk of high endotoxin levels. However PSA preparations with a narrow size distribution and low polydispersity, which are also endotoxin-free, can now be manufactured. Polysaccharide compounds of particular use for the invention are, in one aspect, those produced by bacteria. Some of these naturally-occurring polysaccharides are known as glycolipids. In one embodiment, the polysaccharide compounds are substantially free of terminal galactose units.

### Methods of Attachment

A therapeutic protein may be covalently linked to the polysaccharide compounds by any of various techniques known to those of skill in the art. In various aspects of the invention, sialic acid moieties are bound to a therapeutic protein, e.g., FIX, FVIII, FVIIa or VWF, for example by the method described in U.S. Pat. No. 4,356,170, which is herein incorporated by reference. Additional recent examples include U.S. Pat. No. 7,645,860; U.S. Pat. No. 8,637,640; U.S. Pat. No. 8,642,737; and U.S. Pat. No. 8,809,501, which are herein incorporated by reference.

Other techniques for coupling PSA to polypeptides are also known and contemplated by the invention. For example, US Publication No. 2007/0282096 describes conjugating an amine or hydrazide derivative of, e.g., PSA, to proteins. In addition, US Publication No. 2007/0191597 describes PSA derivatives containing an aldehyde group for reaction with substrates (e.g., proteins) at the reducing end. These references are incorporated by reference in their entireties.

Various methods are disclosed at column 7, line 15, through column 8, line 5 of U.S. Pat. No. 5,846,951 (incorporated by reference in its entirety). Exemplary techniques include linkage through a peptide bond between a carboxyl group on one of either the blood coagulation protein or polysaccharide and an amine group of the blood coagulation protein or polysaccharide, or an ester linkage between a carboxyl group of the blood coagulation protein or polysaccharide and a hydroxyl group of the therapeutic protein or polysaccharide. Another linkage by which the therapeutic protein is covalently bonded to the polysaccharide compound is via a Schiff base, between a free amino group on the blood coagulation protein being reacted with an aldehyde group formed at the non-reducing end of the polysaccharide by periodate oxidation (Jennings H J and Lugowski C, J Immunol. 1981; 127:1011-8; Fernandes A I and Gregoriadis G, Biochim Biophys Acta. 1997; 1341; 26-34). The generated Schiff base is in one aspect stabilized by specific reduction with NaCNBH₃ to form a secondary amine. An alternative approach is the generation of terminal free amino groups in the PSA by reductive amination with NH₄Cl after prior oxidation. Bifunctional reagents can be used for linking two amino or two hydroxyl groups. For example, PSA containing an amino group is coupled to amino groups of the protein with reagents like BS3 (Bis(sulfosuccinimidyl)suberate/Pierce, Rockford, Ill.). In addition heterobifunctional cross linking reagents like sulfo-EMCS (N-ε-maleimidocaproyloxy) sulfosuccinimide ester/Pierce) is used for instance to link amine and thiol groups.

In an exemplary embodiment, a PSA hydrazide is prepared and coupled to the carbohydrate moiety of the protein after prior oxidation and generation of aldehyde functions.

As described above, a free amine group of the therapeutic protein reacts with the 1-carboxyl group of the sialic acid residue to form a peptidyl bond or an ester linkage is formed between the 1-carboxylic acid group and a hydroxyl or other suitable active group on a blood coagulation protein. Alternatively, a carboxyl group forms a peptide linkage with deacetylated 5-amino group, or an aldehyde group of a molecule of a therapeutic protein forms a Schiff base with the N-deacetylated 5-amino group of a sialic acid residue.

Alternatively, the polysaccharide compound is associated in a non-covalent manner with a therapeutic protein. For example, the polysaccharide compound and the pharmaceutically active compound are in one aspect linked via hydrophobic interactions. Other non-covalent associations include electrostatic interactions, with oppositely charged ions attracting each other.

In various embodiments, the therapeutic protein is linked to or associated with the polysaccharide compound in stoichiometric amounts (e.g., 1:1, 1:2, 1:3, 1:4, 1:5, 1:6, 1:7, 1:7, 1:8, 1:9, or 1:10, etc.). In various embodiments, 1-6, 7-12 or 13-20 polysaccharides are linked to the blood coagulation protein. In still other embodiments, 1, 2, 3, 4, 5, 6, 7, 8, 9, 10, 11, 12, 13, 14, 15, 16, 17, 18, 19, 20 or more polysaccharides are linked to the blood coagulation protein.

In various embodiments, the therapeutic protein is modified to introduce glycosylation sites (i.e., sites other than the native glycosylation sites). Such modification may be accomplished using standard molecular biological techniques known in the art. Moreover, the therapeutic protein, prior to conjugation to a water soluble polymer via one or more carbohydrate moieties, may be glycosylated in vivo or in vitro. These glycosylated sites can serve as targets for conjugation of the proteins with water soluble polymers (US Patent Application No. 20090028822, US Patent Application No. 2009/0093399, US Patent Application No. 2009/0081188, US Patent Application No. 2007/0254836, US Patent Application No. 2006/0111279, and DeFrees S. et al., Glycobiology, 2006, 16, 9, 833-43). For example, a protein that is not naturally glycoslyated in vivo (e.g., a protein that is not a glycoprotein) may be modified as described above.

### Aminooxy Linkage

In one embodiment of the invention, the reaction of hydroxylamine or hydroxylamine derivatives with aldehydes (e.g., on a carbohydrate moiety following oxidation by sodium periodate) to form an oxime group is applied to the preparation of conjugates of blood coagulation protein. For example, a glycoprotein (e.g., a therapeutic protein according to the present invention) is first oxidized with a oxidizing agent such as sodium periodate (NaIO₄) (Rothfus J A et Smith E L., J Biol Chem 1963, 238, 1402-10; and Van Lenten L and Ashwell G., J Biol Chem 1971, 246, 1889-94). The periodate oxidation of glycoproteins is based on the classical Malaprade reaction described in 1928, the oxidation of vicinal diols with periodate to form an active aldehyde group (Malaprade L., Analytical application, Bull Soc Chim France, 1928, 43, 683-96). Additional examples for such an oxidizing agent are lead tetraacetate (Pb(OAc)4), manganese acetate (MnO(Ac)3), cobalt acetate (Co(OAc)2), thallium acetate (TlOAc), cerium sulfate (Ce(SO4)2) (U.S. Pat. No. 4,367,309) or potassium perruthenate (KRuO4) (Marko et al., J Am Chem Soc 1997, 119, 12661-2). By “oxidizing agent” a mild oxidizing compound which is capable of oxidizing vicinal diols in carbohydrates, thereby generating active aldehyde groups under physiological reaction conditions is meant.

The second step is the coupling of the polymer containing an aminooxy group to the oxidized carbohydrate moiety to form an oxime linkage. In one embodiment of the invention, this step can be carried out in the presence of catalytic amounts of the nucleophilic catalyst aniline or aniline derivatives (Dirksen A et Dawson P E, Bioconjugate Chem. 2008; Zeng Y et al., Nature Methods 2009, 6, 207-9). The aniline catalysis dramatically accelerates the oxime ligation allowing the use of very low concentrations of the reagents. In an exemplary embodiment of the invention the oxime linkage is stabilized by reduction with NaCNBH₃ to form an alkoxyamine linkage. Additional catalysts are described below.

Additional information on aminooxy technology can be found in the following references, each of which is incorporated in their entireties: EP 1681303A1 (HASylated erythropoietin); WO 2005/014024 (conjugates of a polymer and a protein linked by an oxime linking group); WO96/40662 (aminooxy-containing linker compounds and their application in conjugates); WO 2008/025856 (Modified proteins); Peri F et al., Tetrahedron 1998, 54, 12269-78; Kubler-Kielb J et. Pozsgay V., J Org Chem 2005, 70, 6887-90; Lees A et al., Vaccine 2006, 24(6), 716-29; and Heredia K L et al., Macromoecules 2007, 40(14), 4772-9.

Numerous methods of coupling a water-soluble polymer to an aminooxy linker are contemplated by the present disclosure. For example, coupling of a linker to either the reducing or non-reducing end of a water-soluble polymer such as PSA is described herein. The coupling site (e.g., reducing end versus non-reducing end) is determined by one or more conditions (e.g., time and temperature) of the coupling process as well as the state (e.g., native versus oxidized) of the water-soluble polymer. In one embodiment, an oxidized water-soluble polymer such as PSA is coupled at its non-reducing end to an aminooxy linker by performing the coupling reaction at a reduced temperature (e.g., between 2-8° C.). In an exemplary embodiment, a native (e.g., non-oxidized) water-soluble polymer such as PSA is coupled at it's reducing end to an aminooxy linker by performing the coupling reaction at a higher temperature (e.g., between 22-37° C.). The aforementioned embodiments are described in more detail below and in the Examples.

As described herein, the reaction of oxidized PSA with a diaminooxy linker shows two reactions: a “quick reaction” of the aldehyde group at the non-reducing end, and a “slow reaction” at the reducing end. If native PSA (which is not oxidized and does not contain an active aldehyde group) is reacted with the reducing end at room temperature, a derivatized PSA can be observed. Thus, in various embodiments, in order to minimize an unwanted side reaction at the reducing end of a water-soluble polymer such as PSA, the PSA-aminooxy linker reagent preparation is performed at a temperature between 2-8° C.

In an exemplary embodiment of the present disclosure, the derivatization of native PSA at the reducing end is provided. As described herein, native PSA (which is not oxidized by NaIO₄ and thus does not contain a free aldehyde group at its non-reducing end) is reacted with a diaminooxy linker at room temperature, a derivatization of the PSA at its reducing end can be observed. This coupling occurs through ring opening at the reducing end and subsequent oxime formation (the actual side reaction described above and the cause for the presence of by-product in the aminooxy-PSA reagent). In an exemplary embodiment, the reaction is performed with native PSA yielding in degree of modification of up to about 20, about 21, about 22, about 23, about 24, about 25, about 26, about 27, about 28, about 29, about 30, about 31, about 32, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98 or about 99 percent (%). In an exemplary embodiment the reaction is performed with native PSA yielding in degree of modification of up to about 70%.

In an exemplary embodiment, the reaction is performed with native PSA yielding in degree of modification of about 20% to about 99% and the PSA-rFVIII conjugate obtains a specific activity about 20% to about 150% greater relative to native rFVIII. In an exemplary embodiment, the reaction is performed with native PSA yielding in degree of modification of about 30% to about 90% and the PSA-rFVIII conjugate obtains a specific activity about 30% to about 140% greater relative to native rFVIII. In an exemplary embodiment, the reaction is performed with native PSA yielding in degree of modification of about 30% to about 80% and the PSA-rFVIII conjugate obtains a specific activity about 40% to about 130% greater relative to native rFVIII. In an exemplary embodiment, the reaction is performed with native PSA yielding in degree of modification of about 30% to about 70% and the PSA-rFVIII conjugate obtains a specific activity about 50% to about 120% greater relative to native rFVIII. In an exemplary embodiment, the reaction is performed with native PSA yielding in degree of modification of about 30% to about 60% and the PSA-rFVIII conjugate obtains a specific activity about 40% to about 110% greater relative to native rFVIII. In an exemplary embodiment, the reaction is performed with native PSA yielding in degree of modification of up to about 35% and the PSA-rFVIII conjugate obtains a specific activity of about 50% to about 120% greater relative to native rFVIII. In an exemplary embodiment, the reaction is performed with native PSA yielding in degree of modification of up to about 54% and the PSA-rFVIII conjugate obtains a specific activity of about 50% to about 120% greater relative to native rFVIII. In an exemplary embodiment, the reaction is performed with native PSA yielding in degree of modification of up to about 58% and the PSA-rFVIII conjugate obtains a specific activity of about 50% to about 120% greater relative to native rFVIII. In an exemplary embodiment, the reaction is performed with native PSA yielding in degree of modification of up to about 70% and the PSA-rFVIII conjugate obtains a specific activity of about 70% greater relative to native rFVIII.

As a main product the following structure was determined by 13C NMR spectroscopy

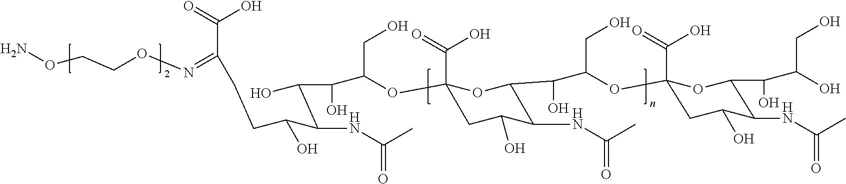

In an exemplary embodiment, PSAs and mPSAs comprise at least 2, preferably at least 5, more preferably at least 10 and most preferably at least 20 N-acetylneuraminic acid moieties (n). In an exemplary embodiment, n may comprise from 2 to 300 N-acetylneuraminic acid moieties. In an exemplary embodiment, n may comprise from 5 to 200 N-acetylneuraminic acid moieties. In an exemplary embodiment, n may comprise from 10 to 100 N-acetylneuraminic acid moieties. The reaction can be transferred to other carbohydrates like dextran and starch or other polysaccharides containing reducing end groups. The use of a nucleophilic catalyst like m-toluidine or aniline is also contemplated. Thus, preparation of aminooxy-PSA reagents using native PSA (i.e. without prior oxidation), which can then be used for chemical modification of therapeutic proteins, is provided herein.

Thus, in various embodiments of the present disclosure, methods are provided wherein the conditions of coupling, (e.g., 2-8° C. incubation temperature) a diaminooxy linker to a water soluble polymer such as oxidized PSA, favor the coupling to either the non-reducing end or, in one alternative, wherein the conditions of coupling (e.g., room temperature incubation) a diaminooxy linker to a water soluble polymer such as native, non-oxidized PSA, favor the coupling to either the reducing end.

In various embodiments of the invention, the water soluble polymer which is linked according to the aminooxy technology described herein to an oxidized carbohydrate moiety of a therapeutic protein (e.g., FVIII, FVIIa, or FIX) include, but are not limited to polyethylene glycol (PEG), branched PEG, polysialic acid (PSA), carbohydrate, polysaccharides, pullulane, chitosan, hyaluronic acid, chondroitin sulfate, dermatan sulfate, starch, dextran, carboxymethyl-dextran, polyalkylene oxide (PAO), polyalkylene glycol (PAG), polypropylene glycol (PPG) polyoxazoline, poly acryloylmorpholine, polyvinyl alcohol (PVA), polycarboxylate, polyvinylpyrrolidone, polyphosphazene, polyoxazoline, polyethylene-co-maleic acid anhydride, polystyrene-co-maleic acid anhydride, poly(l-hydroxymethylethylene hydroxymethylformal) (PHF), 2-methacryloyloxy-2′-ethyltrimethylammoniumphosphate (MPC).

### Nucleophilic Catalysts

As described herein, the conjugation of water soluble polymers to therapeutic proteins can be catalyzed by aniline. Aniline strongly catalyzes aqueous reactions of aldehydes and ketones with amines to form stable imines such as hydrazones and oximes. The following diagram compares an uncatalyzed versus the aniline-catalyzed oxime ligation reaction (Kohler J J, Chem Bio Chem 2009, 10, 2147-50):

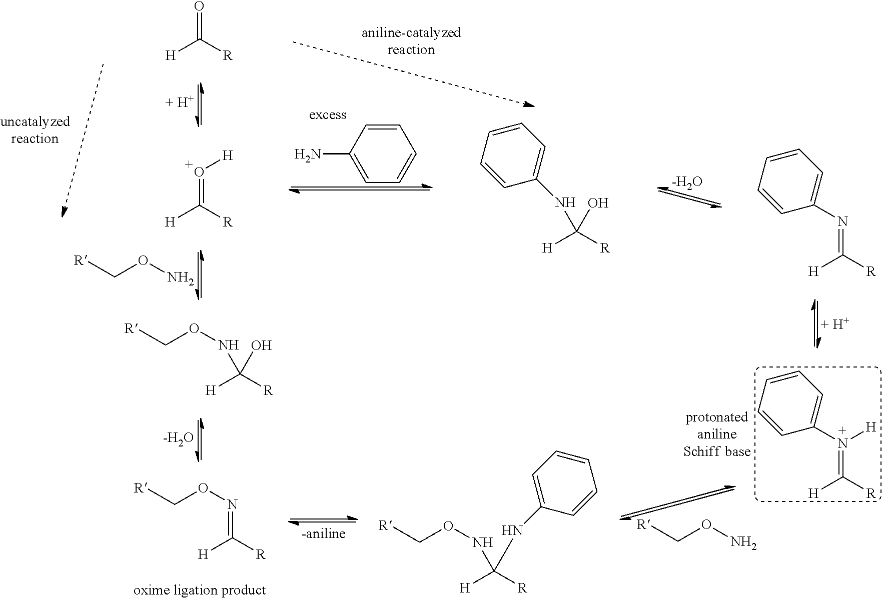

However, considering the numerous health risks associated with aniline, alternative catalysts are desirable. The present invention provides aniline derivatives as alternative oxime ligation catalysts. Such aniline derivatives include, but are not limited to, o-amino benzoic acid, m-amino benzoic acid, p-amino benzoic acid, sulfanilic acid, o-aminobenzamide, o-toluidine, m-toluidine, p-toluidine, o-anisidine, m-anisidine, and p-anisidine.

In one embodiment of the invention, m-toluidine (aka meta-toluidine, m-methylaniline, 3-methylaniline, or 3-amino-1-methylbenzene) is used to catalyze the conjugation reactions described herein. m-toluidine and aniline have similar physical properties and essentially the same pKa value (m-toluidine: pKa 4.73, aniline: pKa 4.63).

The nucleophilic catalysts of the invention are useful for oxime ligation (e.g, using aminooxy linkage) or hydrazone formation (e.g., using hydrazide chemistry). In various embodiments of the invention, the nucleophilic catalyst is provided in the conjugation reaction at a concentration of about 0.1, about 0.2, about 0.3, about 0.5, about 0.6, about 0.7, about 0.8, about 0.9, about 1.0, about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5, about 10, about 11, about 12, about 13, about 14, about 15, about 16, about 17, about 18, about 19, about 20, about 25, about 30, about 35, about 40, about 45, or about 50 mM. In one embodiment, the nucleophilic catalyst is provided in an amount from about 1 mM to about 10 mM. In various embodiments of the invention, the pH of conjugation reaction mixture is about 4.5, about 5.0, about 5.5, about 6.0, about 6.5, about 7.0 and about 7.5. In one embodiment, the pH is from about 5.5 to about 6.5.

### Purification of Conjugated Proteins

In various embodiments, purification of a protein that has been incubated with an oxidizing agent and/or a therapeutic protein that has been conjugated with a water soluble polymer according to the present disclosure, is desired. Numerous purification techniques are known in the art and include, without limitation, chromatographic methods such as ion-exchange chromatography, hydrophobic interaction chromatography, size exclusion chromatography and affinity chromatography or combinations thereof, filtration methods (e.g., UF/DF), and precipitation methods as well as dialysis procedures and any combinations of the aforementioned techniques (Guide to Protein Purification, Meth. Enzymology Vol 463 (edited by Burgess R R and Deutscher M P), 2^{nd} edition, Academic Press 2009).

### Efficacy

In various embodiments, murine models can be used to assess half-life efficacy. In various embodiments, murine models can be used to assess half-life efficacy. In an exemplary embodiment, murine models are used to determine the PSA modified FVIII has an in vivo half-life that is longer than a PEGylated FVIII. In an exemplary embodiment, murine models are used to determine a modified FVIII conjugated to a PSA moiety having a mean molecular weight of about 20 kDa has an in vivo half-life that is longer than a PEGylated FVIII, which is conjugated to a PEG moiety having a mean molecular weight of about 20 kDa. In an exemplary embodiment, a tail-clip bleeding model in the FVIII KO mouse is used to determine the PSA modified FVIII has an in vivo half-life that is longer than a PEGylated FVIII. In an exemplary embodiment, a carotid occlusion model in the FVIII KO mouse is used to determine the PSA modified FVIII has an in vivo half-life that is longer than a PEGylated FVIII. In an exemplary embodiment, a murine model of hemophilic joint bleeding (Intra-Articular Puncture) is used to determine the PSA modified FVIII has an in vivo half-life that is longer than a PEGylated FVIII.

The following examples are not intended to be limiting but only exemplary of specific embodiments of the invention.

### EXAMPLES

### Example 1

Preparation of the Homobifunctional Linker NH₂[OCH₂CH₂]₂ONH₂

The homobifunctional linker NH₂[OCH₂CH₂]₂ONH₂

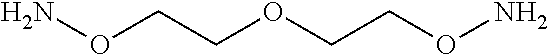

(3-oxa-pentane-1,5-dioxyamine) containing two active aminooxy groups was synthesized according to Boturyn et al. (Tetrahedron 1997, 53, 5485-92) in a two step organic reaction employing a modified Gabriel-Synthesis of primary amines. In the first step, one molecule of 2,2-chlorodiethylether was reacted with two molecules of endo-N-hydroxy-5-norbomene-2,3-dicarboximide in dimethylformamide (DMF). The desired homobifunctional product was prepared from the resulting intermediate by hydrazinolysis in ethanol.

### Example 2

Preparation of the Homobifunctional Linker NH₂[OCH₂CH₂]₄ONH₂

The homobifunctional linker NH₂[OCH₂CH₂]₄ONH₂

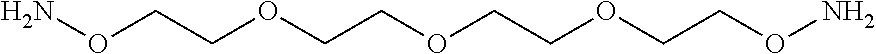 [SMILES: NOCCOCCOCCOCCON]

(3,6,9-trioxa-undecane-1,11-dioxyamine) containing two active aminooxy groups was synthesized according to Boturyn et al. (Tetrahedron 1997, 53, 5485-92) in a two step organic reaction employing a modified Gabriel-Synthesis of primary amines. In the first step one molecule of Bis-(2-(2-chlorethoxy)-ethyl)-ether was reacted with two molecules of Endo-N-hydroxy-5-norbornene-2,3-dicarboximide in DMF. The desired homobifunctional product was prepared from the resulting intermediate by hydrazinolysis in ethanol.

### Example 3

Preparation of the Homobifunctional Linker NH₂[OCH₂CH₂]₆ONH₂

The homobifunctional linker NH₂[OCH₂CH₂]₆ONH₂

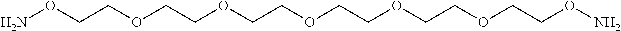 [SMILES: NOCCOCCOCCOCCOCCOCCON]

(3,6,9,12,15-penatoxa-heptadecane-1,17-dioxyamine) containing two active aminooxy groups was synthesized according to Boturyn et al. (Tetrahedron 1997, 53, 5485-92) in a two step organic reaction employing a modified Gabriel-Synthesis of primary amines. In the first step one molecule of hexaethylene glycol dichloride was reacted with two molecules of endo-N-hydroxy-5-norbornene-2,3-dicarboximide in DMF. The desired homobifunctional product was prepared from the resulting intermediate by hydrazinolysis in ethanol.

### Example 4

### Detailed Synthesis of an Aminooxy-PSA Reagent

3-oxa-pentane-1,5 dioxyamine was synthesized according to Botyryn et al (Tetrahedron 1997; 53:5485-92) in a two step organic synthesis as outlined in Example 1.

### Step 1:

To a solution of Endo-N-hydroxy-5-norbonene-2,3-dicarboxiimide (59.0 g; 1.00 eq) in 700 mL anhydrous N,N-dimetylformamide anhydrous K₂CO₃ (45.51 g; 1.00 eq) and 2,2-dichlorodiethylether (15.84 mL; 0.41 eq) were added. The reaction mixture was stirred for 22 h at 50° C. The mixture was evaporated to dryness under reduced pressure. The residue was suspended in 2 L dichloromethane and extracted two times with saturated aqueous NaCl-solution (each 1 L). The Dichloromethane layer was dried over Na₂SO₄ and then evaporated to dryness under reduced pressure and dried in high vacuum to give 64.5 g of 3-oxapentane-1,5-dioxy-endo-2′,3′-dicarboxydiimidenorbornene as a white-yellow solid (intermediate 1).

### Step 2:

To a solution of intermediate 1 (64.25 g; 1.00 eq) in 800 mL anhydrous Ethanol, 31.0 mL Hydrazine hydrate (4.26 eq) were added. The reaction mixture was then refluxed for 2 h. The mixture was concentrated to the half of the starting volume by evaporating the solvent under reduced pressure. The occurring precipitate was filtered off. The remaining ethanol layer was evaporated to dryness under reduced pressure. The residue containing the crude product 3-oxa-pentane-1,5-dioxyamine was dried in vacuum to yield 46.3 g. The crude product was further purified by column chromatography (Silicagel 60; isocratic elution with Dichloromethane/Methanol mixture, 9/1) to yield 11.7 g of the pure final product 3-oxa-pentane-1,5-dioxyamine.

### Example 5

### Preparation of Aminooxy-PSA

1000 mg of oxidized PSA (MW=20 kD) obtained from the Serum Institute of India (Pune, India) was dissolved in 16 mL 50 mM phosphate buffer pH 6.0. Then 170 mg 3-oxa-pentane-1,5-dioxyamine was given to the reaction mixture. After shaking for 2 h at RT 78.5 mg sodium cyanoborohydride was added and the reaction was performed for 18 hours over night. The reaction mixture was then subjected to an ultrafiltration/diafiltration procedure (UF/DF) using a membrane with a 5 kD cut-off made of regenerated cellulose (50 cm², Millipore).

### Example 6

### Preparation of Aminooxy-PSA Employing a Chromatographic Purification Step

1290 mg of oxidized PSA (MW=20 kD) obtained from the Serum Institute of India (Pune, India) was dissolved in 25 mL 50 mM phosphate buffer pH 6.0 (Buffer A). Then 209 mg 3-oxa-pentane-1,5-dioxyamine was given to the reaction mixture. After shaking for 1 h at RT 101 mg sodium cyanoborohydride was added and the reaction was performed for 3 hours. Then the mixture was then subjected to a weak anion exchange chromatography step employing a Fractogel EMD DEAE 650-M chromatography gel (column dimension: XK26/135). The reaction mixture was diluted with 110 mL Buffer A and loaded onto the DEAE column pre-equilibrated with Buffer A at a flow rate of 1 cm/min. Then the column was washed with 20 CV Buffer B (20 mM Hepes, pH 6.0) to remove free 3-oxa-pentane-1,5-dioxyamine and cyanide at a flow rate of 2 cm/min. The aminooxy-PSA reagent was then eluted with a step gradient consisting of 67% Buffer B and 43% Buffer C (20 mM Hepes, 1M NaCl, pH 7.5). The eluate was concentrated by UF/DF using a 5 kD membrane made of polyether sulfone (50 cm², Millipore). The final diafiltration step was performed against Buffer D (20 mM Hepes, 90 mM NaCl, pH 7.4). The preparation was analytically characterized by measuring total PSA (Resorcinol assay) and total aminooxy groups (TNBS assay) to determine the degree of modification. Furthermore the polydispersity as well as free 3-oxa-pentane-1,5-dioxyamine and cyanide was determined.

### Example 7

Preparation of Aminooxy-PSA without a Reduction Step

573 mg of oxidized PSA (MW=20 kD) obtained from the Serum Institute of India (Pune, India) was dissolved in 11.3 mL 50 mM phosphate buffer pH 6.0 (Buffer A). Then 94 mg 3-oxa-pentane-1,5-dioxyamine was given to the reaction mixture. After shaking for 5 h at RT the mixture was then subjected to a weak anion exchange chromatography step employing a Fractogel EMD DEAE 650-M chromatography gel (column dimension: XK16/105). The reaction mixture was diluted with 50 mL Buffer A and loaded onto the DEAE column pre-equilibrated with Buffer A at a flow rate of 1 cm/min. Then the column was washed with 20 CV Buffer B (20 mM Hepes, pH 6.0) to remove free 3-oxa-pentane-1,5-dioxyamine and cyanide at a flow rate of 2 cm/min. The aminooxy-PSA reagent was the eluted with a step gradient consisting of 67% Buffer B and 43% Buffer C (20 mM Hepes, 1 M NaCl, pH 7.5). The eluate was concentrated by UF/DF using a 5 kD membrane made of polyether sulfone (50 cm², Millipore). The final diafiltration step was performed against Buffer D (20 mM Hepes, 90 mM NaCl, pH 7.4). The preparation was analytically characterized by measuring total PSA (Resorcinol assay) and total aminooxy groups (TNBS assay) to determine the degree of modification. Furthermore the polydispersity as well as free 3-oxa-pentane-1,5-dioxyamine was determined.

### Example 8

Preparation of Aminooxy-PSA without a Reduction Step in the Presence of the Nucleophilic Catalyst m-Toluidine

573 mg of oxidized PSA (MW=20 kD) obtained from the Serum Institute of India (Pune, India) is dissolved in 9 mL 50 mM phosphate buffer pH 6.0 (Buffer A). Then 94 mg 3-oxa-pentane-1,5-dioxyamine is given to this solution. Subsequently 2.3 mL of a 50 mM m-toluidine stock solution are added to this reaction mixture. After shaking for 2 h at RT the mixture is then subjected to a weak anion exchange chromatography step employing a Fractogel EMD DEAE 650-M chromatography gel (column dimension: XK16/105). The reaction mixture is diluted with 50 mL Buffer A and loaded onto the DEAE column pre-equilibrated with Buffer A at a flow rate of 1 cm/min. Then the column is washed with 20CV Buffer B (20 mM Hepes, pH 6.0) to remove free 3-oxa-pentane-1,5-dioxyamine and cyanide at a flow rate of 2 cm/min. The aminooxy-PSA reagent is the eluted with a step gradient consisting of 67% Buffer B and 43% Buffer C (20 mM Hepes, 1 M NaCl, pH 7.5). The eluate is concentrated by UF/DF using a 5 kD membrane made of polyether sulfone (50 cm², Millipore). The final diafiltration step is performed against Buffer D (20 mM Hepes, 90 mM NaCl, pH 7.4). The preparation is analytically characterized by measuring total PSA (Resorcinol assay) and total aminooxy groups (TNBS assay) to determine the degree of modification. Furthermore the polydispersity as well as free 3-oxa-pentane-1,5-dioxyamine is determined.

### Example 9

### Preparation of Aminooxy-PSA Reagent

An Aminooxy-PSA reagent was prepared according to the Examples 4-8. After diafiltration, the product was frozen at −80° C. and lyophilized. After lyophilization the reagent was dissolved in the appropriate volume of water and used for preparation of PSA-protein conjugates via carbohydrate modification.

### Example 10

Polysialylation of rFVIII Using Aminooxy-PSA and m-Toluidine as a Nucleophilic Catalyst

### Method 1:

50 mg rFVIII was transferred into reaction buffer (50 mM Hepes, 350 mM sodium chloride, 5 mM calcium chloride, pH 6.0) and diluted to obtain a protein concentration of 1 mg/mL. To this solution, NaIO₄ was added to give a final concentration of 200 μM. The oxidation was carried at RT for 30 min in the dark under gentle shaking. Then the reaction was quenched with cysteine (final concentration: 10 mM) for 60 min at RT. The solution was subjected to an IEX column with a volume of 20 mL (Merck EMD TMAE (M)) which was equilibrated with Buffer A (20 mM Hepes, 5 mM CaCl₂, pH 7.0). The column was equilibrated with 5 CV Buffer A. Then the oxidized rFVIII was eluted with Buffer B (20 mM Hepes, 5 mM CaCl₂, 1M NaCl, pH 7.0). The rFVIII containing fractions were collected. The protein content was determined (Coomassie, Bradford) and adjusted to 1 mg/mL with reaction buffer and adjusted to pH 6.0 by dropwise addition of 0.5 M HCl. Then a 50-fold molar excess of a aminooxy-PSA reagent with a MW of 20 kD (described above) was added followed by m-toluidine as a nucleophilic catalyst (final concentration: 10 mM). The coupling reaction was performed for 2 hours in the dark under gentle shaking at room temperature. The excess of aminooxy-PSA reagent was removed by means of HIC. The conductivity of the reaction mixture was raised to 130 mS/cm by adding a buffer containing ammonium acetate (50 mM Hepes, 350 mM sodium chloride, 5 mM calcium chloride, 8 M ammonium acetate, pH 6.9) and loaded onto a column filled with 80 mL Phenyl Sepharose FF (GE Healthcare, Fairfield, Conn.) pre-equilibrated with 50 mM Hepes, 2.5 M ammonium acetate, 350 mM sodium chloride, 5 mM calcium chloride, pH 6.9. Subsequently, the conjugate was eluted with 50 mM Hepes buffer pH 7.5 containing 5 mM CaCl₂. Finally, the PSA-rFVIII containing fractions were collected and subjected to UF/DF by use of a 30 kD membrane made of regenerated cellulose (88 cm², Millipore). The preparation was analytically characterized by measuring total protein (Coomassie, Bradford) and FVIII chromogenic activity. The PSA-rFVIII conjugate showed a specific activity of >70% in comparison to native rFVIII was determined.

### Method 2:

58 mg of recombinant factor VIII (rFVIII) in Hepes buffer (50 mM HEPES, ˜350 mM sodium chloride, 5 mM calcium chloride, 0.1% Polysorbate 80, pH 7.4) is dissolved in reaction buffer (50 mM Hepes, 350 mM sodium chloride, 5 mM calcium chloride, pH 6.0) to get a final protein concentration of 1.0+/−0.25 mg/mL. Then the pH of the solution is corrected to 6.0 by drop wise addition of a 0.5 N aqueous HCl solution. Subsequently, a 40 mM aqueous sodium periodate solution is added within 10 minutes to give a concentration of 200 μM. The oxidation reaction is carried out for 30+/−5 min at a temperature (T) of T=+22+/−2° C. Then the reaction is stopped by addition of an aqueous L-cysteine solution (1 M) within 15 minutes at T=+22+/−2° C. to give a final concentration of 10 mM in the reaction mixture and incubation for 60+/−5 min.

The oxidized rFVIII is further purified by anion exchange chromatography on EMD TMAE (M) (Merck). The mixture is diluted with Buffer A (20 mM Hepes, 5 mM CaCl₂, pH 6.5) to give a conductivity of 5 ms/cm. This solution is loaded onto the IEX column (bed height: 5.4 cm) with a column volume of 10 mL using a flow rate of 1.5 cm/min. This column is subsequently washed (flow rate: 1.5 cm/min) with 5 CV of a 92:8 mixture (w/w) of Buffer A and Buffer B (20 mM Hepes, 5 mM CaCl₂, 1.0 M NaCl, pH 7.0). Then the oxidized rFVIII is eluted with a 50:50 (w/w) mixture of Buffer A and Buffer B followed by a postelution step with 5 CV of Buffer B. The elution steps are carried out by use of a flow rate of 1.0 cm/min.

Subsequently, the aminooxy-polysialic acid (PSA-ONH₂) reagent is added in a 50-fold molar excess to the eluate containing the purified oxidized rFVIII within a maximum time period (t) of 15 minutes under gentle stirring. Then an aqueous m-toluidine solution (50 mM) is added within 15 minutes to get a final concentration of 10 mM. The reaction mixture is incubated for 120+/−10 min. in the dark at a temperature (T) of T=+22+/−2° C. under gentle shaking.

The obtained PSA-rFVIII conjugate is purified by Hydrophobic Interaction Chromatography (HIC) using a Phenyl Sepharose FF low sub resin (GE Healthcare) packed into a column manufactured by GE Healthcare with a bed height (h) of 15 cm and a resulting column volume (CV) of 81 mL.

The reaction mixture is spiked with ammonium acetate by addition of 50 mM Hepes buffer, containing 350 mM sodium chloride, 8 M ammonium acetate, 5 mM calcium chloride, pH 6.9. Two volumes of the reaction mixture are mixed with 1 volume of the ammonium acetate containing buffer system and the pH value is corrected to pH 6.9 by drop wise addition of a 0.5 N aqueous NaOH solution. This mixture is loaded onto the HIC column at flow rate of 1 cm/min followed by a washing step using >3 CV equilibration buffer (50 mM Hepes, 350 mM sodium chloride, 2.5 M ammonium acetate, 5 mM calcium chloride, pH 6.9).

For removal of reaction by-products and anti-chaotropic salt a second washing step is performed with >5 CV washing buffer 1 (50 mM Hepes, 3 M sodium chloride, 5 mM calcium chloride, pH 6.9) in upflow mode at a flow rate of 2 cm/min. Then elution of purified PSA-rFVIII conjugate is performed in down flow mode using a step gradient of 40% washing buffer 2 (50 mM Hepes, 1.5 M sodium chloride, 5 mM calcium chloride, pH 6.9) and 60% elution buffer (20 mM Hepes, 5 mM calcium chloride, pH 7.5) at a flow rate of 1 cm/min. The elution of the PSA-rFVIII conjugate is monitored at UV 280 nm and the eluate containing the conjugate is collected within <4 CV. The post elution step is performed with >3 CV elution buffer under the same conditions to separate minor and/or non modified rFVIII from the main product.

Finally the purified conjugate is concentrated by ultra-/diafiltration (UF/DF) using a membrane made of regenerated cellulose with a molecular weight cut off 30 kD (88 cm², Millipore).

The conjugate prepared by use of this procedure are analytically characterized by measuring total protein, FVIII chromogenic activity and determination of the polysialyation degree by measuring the PSA content (resorcinol assay). For the conjugate obtained a specific activity >50% and a PSA degree >5.0 is calculated.

### Method 3:

50 mg rFVIII was transferred into reaction buffer (50 mM Hepes, 350 mM sodium chloride, 5 mM calcium chloride, pH 6.0) and diluted to obtain a protein concentration of 1 mg/mL. A 50-fold molar excess of aminooxy-PSA reagent with a MW of 20 kD (described above) was added followed by m-toluidine as a nucleophilic catalyst (final concentration: 10 mM) and NaIO₄ (final concentration: 400 μM). The coupling reaction was performed for 2 hours in the dark under gentle shaking at room temperature. Subsequently, the reaction was quenched with cysteine for 60 min at RT (final concentration: 10 mM). Then the conductivity of the reaction mixture was raised to 130 mS/cm by adding a buffer containing ammonium acetate (50 mM Hepes, 350 mM sodium chloride, 5 mM calcium chloride, 8 M ammonium acetate, pH 6.9) and loaded onto a column filled with 80 mL Phenyl Sepharose FF (GE Healthcare, Fairfield, Conn.) pre-equilibrated with 50 mM Hepes, 2.5 M ammonium acetate, 350 mM sodium chloride, 5 mM calcium chloride, 0.01% Tween 80, pH 6.9. Subsequently, the conjugate was eluted with 50 mM Hepes, 5 mM calcium chloride, pH 7.5. Finally, the PSA-rFVIII containing fractions were collected and subjected to UF/DF by use of a 30 kD membrane made of regenerated cellulose (88 cm², Millipore). The preparation was analytically characterized by measuring total protein (Bradford) and FVIII chromogenic activity. For the PSA-rFVIII conjugate a specific activity of ≧70% in comparison to native rFVIII was determined.

### Method 4:

50 mg recombinant factor VIII (rFVIII) in 50 mM Hepes buffer (50 mM HEPES, ˜350 mM sodium chloride, 5 mM calcium chloride, 0.1% Polysorbate 80, pH 7.4) was dissolved in reaction buffer (50 mM Hepes, 350 mM sodium chloride, 5 mM calcium chloride, pH 6.0) to get a final protein concentration of 1.0+/−0.25 mg/mL. Then the pH of the solution was corrected to 6.0 by drop wise addition of a 0.5 N aqueous HCl solution.

Subsequently, the aminooxy-polysialic acid (PSA-ONH₂) reagent was added in a 50-fold molar excess to this rFVIII solution within a maximum time period (t) of 15 minutes under gentle stirring. Then an aqueous m-toluidine solution (50 mM) was added within 15 minutes to get a final concentration of 10 mM. Finally, a 40 mM aqueous sodium periodate solution was added to give a concentration of 400 μM.

The reaction mixture was incubated for 120+/−10 min. in the dark at a temperature (T) of T=+22+/−2° C. under gentle shaking. Then the reaction was stopped by the addition of an aqueous L-cysteine solution (1 M) to give a final concentration of 10 mM in the reaction mixture and incubation for 60+/−5 min.

The obtained PSA-rFVIII conjugate was purified by Hydrophobic Interaction Chromatography (HIC) using a Phenyl Sepharose FF low sub resin (GE Healthcare) packed into a column manufactured by GE Healthcare with a bed height (h) of 15 cm and a resulting column volume (CV) of 81 mL.

The reaction mixture was spiked with ammonium acetate by addition of 50 mM Hepes buffer, containing 350 mM sodium chloride, 8 M ammonium acetate, 5 mM calcium chloride, pH 6.9. Two volumes of the reaction mixture was mixed with 1 volume of the ammonium acetate containing buffer system and the pH value was corrected to pH 6.9 by drop wise addition of an 0.5 N aqueous NaOH solution. This mixture was loaded onto the HIC column using a flow rate of 1 cm/min followed by a washing step using >3 CV equilibration buffer (50 mM Hepes, 350 mM sodium chloride, 2.5 M ammonium acetate, 5 mM calcium chloride, pH 6.9).

For removal of reaction by-products and anti-chaotropic salt a second washing step was performed with >5CV washing buffer 1 (50 mM Hepes, 3 M sodium chloride, 5 mM calcium chloride, pH 6.9) in upflow mode at a flow rate of 2 cm/min. Then elution of purified rFVIII conjugate was performed in down flow mode using a step gradient of 40% washing buffer 2 (50 mM Hepes, 1.5 M sodium chloride, 5 mM calcium chloride, pH 6.9) and 60% elution buffer (20 mM Hepes, 5 mM calcium chloride, pH 7.5) at a flow rate of 1 cm/min. The elution of the PSA-rFVIII conjugate was monitored at UV 280 nm and the eluate containing the conjugate was collected within <4 CV. The post elution step was performed with >3 CV elution buffer under the same conditions to separate minor and/or non modified rFVIII from the main product.

Finally, the purified conjugate was concentrated by ultra-/diafiltration (UF/DF) using a membrane made of regenerated cellulose with a molecular weight cut off 30 kD (88 cm², Millipore).

The conjugates prepared by use of this procedure were analytically characterized by measuring total protein, FVIII chromogenic activity and determination of the polysialyation degree by measuring the PSA content (resorcinol assay).
Analytical data (mean of 6 consecutive batches):
Process yield (Bradford): 58.9%
Process yield (FVIII chrom.): 46.4%
Specific activity: (FVIII chrom./mg protein): 4148 IU/mg
Specific activity (% of starting material): 79.9%
PSA degree (mol/mol): 8.1

### Example 11

### Coupling of a Diamonooxy Linker to Native PSA

This Example describes procedures to prepare aminooxy-PSA reagents using native PSA (i.e. without prior oxidation), which can be used for chemical modification of therapeutic proteins.

### a) Coupling at Ambient Temperature

52.2 mg of native PSA (MW=20 kD) obtained from the Serum Institute of India (Pune, India) was dissolved in 1.05 mL 50 mM phosphate buffer pH 6.0. Then 10.3 mg 3-oxa-pentane-1,5-dioxyamine (linker molecule) was added drop wise to the reaction mixture. The reaction was incubated for 2 h at room temperature under gentle agitation in the dark.

### b) Coupling at Increased Temperature

52.2 mg of native PSA (MW=20 kD) obtained from the Serum Institute of India (Pune, India) was dissolved in 1.05 mL 50 mM phosphate buffer pH 6.0. Then 10.3 mg 3-oxa-pentane-1,5-dioxyamine (linker molecule) was added drop wise to the reaction mixture. The reaction was incubated for 2 h at room temperature under gentle agitation in the dark. Then the temperature was increased to 32-37° C. and the reaction mixture was incubated for another 14 h.

### c) Coupling at Increased Temperature and Increased Linker Excess

52.2 mg of native PSA (MW=20 kD) obtained from the Serum Institute of India (Pune, India) was dissolved in 1.05 mL 50 mM phosphate buffer pH 6.0. Then 10.3 mg 3-oxa-pentane-1,5-dioxyamine (linker molecule) was added drop wise to the reaction mixture. The reaction was incubated for 2 h at room temperature under gentle agitation in the dark. Then 26.3 mg 3-oxa-pentane-1,5-dioxyamine were added drop wise to the reaction, the temperature was increased to 32-37° C. and the reaction mixture was incubated for another 14 h.

### d) Purification of PSA Derivatives

After the incubation was completed, the reaction mixtures generated under points a-c were purified by extensive dialysis. Therefore samples of the reaction mixtures were loaded into Slide-A-Lyzer dialysis cassettes (0-5-3 mL, MWCO 3.5 kD, reg. cellulose, Pierce) and dialyzed against 10 mM phosphate buffer pH 8.0 according to the following pattern:

2 h against 500 mL buffer at room temperature

2 h against 500 mL buffer at room temperature

12 h against 500 mL buffer at 4° C.

1 h against 50 mL ‘Slide-A-Lyzer Concentrating Solution for Dialysis’ at room temperature for concentration to initial sample volume.

The purified aminooxy-PSA is thus ready to be used in a protein conjugation reaction according to, for example, Examples 11, 12, 14, and 17-31, above. Likewise, any of the water-soluble polymers described herein can be coupled to an aminooxy linker as described in this Example and then conjugated to a protein as set out in the above Examples.

The preparation was analytically characterized by measuring total PSA (Resorcinol assay) and total aminooxy groups (TNBS assay) to determine the degree of modification. For preparation (a) a modification degree (MD) of 0.35, for (b) MD=0.54 and for (c) MD=0.58 was determined. Furthermore the polydispersity as well as free 3-oxa-pentane-1,5-dioxyamine was measured. The polydispersity was lower than 1.15 for all preparations and the content of free linker was lower than 0.15 mol % of the PSA concentration.

For the PSA modified at the reducing end the following structure was determined by ¹³C NMR spectroscopy.

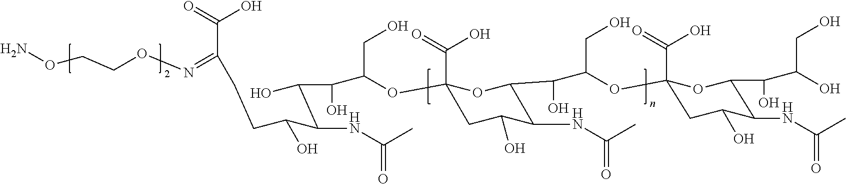

### Example 12

### Preparation of Aminooxy-PSA at 4° C. Employing a Chromatographic Purification Step

During a detailed analytical characterization of the aminooxy-PSA reagent prepared at room temperature, NMR studies (See, e.g., U.S. Provisional Application No. 61/647,814, incorporated by reference in it's entirety) revealed that the derivatization of oxidized PSA with the diaminooxy linker consists of two distinct reactions: a quick reaction of the aldehyde group at the non-reducing end of PSA and a slow reaction of the aldehyde group (in the form of a hemiketal) at the reducing end of PSA. The latter reaction could be considered an unwanted side reaction that is to be avoided for reagent production.

Therefore, the process for the preparation of the aminooxy-PSA reagent has been optimized as described in the instant Example. The reducing end only occurs to a significant degree if the process is performed at room temperature. Hence, the process was adjusted and is conducted at 2-8° C. By performing the whole process (chemical reaction and purification of the PSA reagent by IEX) at 2-8° C., the side reaction at the reducing end of PSA was substantially reduced. This process change thus leads to a reagent of higher quality.

### Procedure

1290 mg of oxidized PSA (MW=20 kD) obtained from the Serum Institute of India (Pune, India) was dissolved in 25 mL 50 mM phosphate buffer pH 6.0 (Buffer A). Then 209 mg 3-oxa-pentane-1,5-dioxyamine was added to the reaction mixture and incubated for 1 h at 2-8° C. under gentle agitation in the dark.

After incubation, the mixture was subjected to a weak anion exchange chromatography step employing a Fractogel EMD DEAE 650-M chromatography gel (column dimension: XK26/135) carried out in a cold room at temperature of 2-8° C. The reaction mixture was diluted with pre-cooled Buffer A (110 mL) and loaded onto the DEAE column pre-equilibrated with Buffer A at a flow rate of 1 cm/min. Then the column was washed with 20 CV Buffer B (20 mM Hepes, pH 6.0) at a flow rate of 2 cm/min to remove free 3-oxa-pentane-1,5-dioxyamine. The aminooxy-PSA reagent was then eluted with a step gradient consisting of 67% Buffer B and 43% Buffer C (20 mM Hepes, 1M NaCl, pH 7.5). The eluate was concentrated by UF/DF using a 5 kD membrane made of polyether sulfone (50 cm2, Millipore). The preparation was analytically characterized by measuring total PSA (Resorcinol assay) and total aminooxy groups (TNBS assay) to determine the degree of modification. The PSA concentration in the final preparation was 46.0 mg/mL and the modification degree was 83.5%. Furthermore, a polydispersity value of 1.131 was determined. In addition a concentration of 0.22 μg/mL (0.07 mol % of PSA) was measured for free 3-oxa-pentane-1,5-dioxyamine.

The purified aminooxy-PSA is thus ready to be used in a conjugation reaction according to Examples 11, 12, 14, and 17-31, above.

### Example 13

Synthesis of Polysialylated rFVIII (Large Scale)

rFVIII was polysialylated in large scale according to the method as outlined in Example 9 with minor modifications. For this purpose, 1.5 g rFVIII was polysialylated in Hepes buffer (50 mM Hepes, 350 mM sodium chloride, 5 mM calcium chloride, pH 6.0) as described (protein concentration: 1.1 mg/mL/determined by fluorescence assay). Then the product was purified by Hydrophobic Interaction Chromatography using a Phenyl Sepharose FF low sub resin (GE Healthcare). Then the eluate was concentrated by ultra-/diafiltration (UF/DF) using a membrane made of regenerated cellulose with a molecular weight cut off of 30 kD. Then the concentrate was applied to a size exclusion chromatography column (Superose 6 prep grade/GE Healthcare). This procedure is used as a final polishing step to separate potential impurities from the product. Finally, the purified conjugate (“PSA-rFVIII”) was concentrated again by UF/DF (regenerated cellulose/molecular weight cut off 30 kD). Using this method BDS was prepared under GMP conditions to manufacture material used for Clinical Phase I Study: LOT D, LOT E, and LOT F.

### Example 14

### Determination of VWF-FVIII Binding Affinity by Surface Plasmon Resonance (SPR)

VWF-FVIII binding affinity was analyzed using a Biacore instrument (GE Healthcare, Uppsala, Sweden) as follows.

Plasma-derived VWF (pdVWF, Diagnostica Stago, Asnieres sur Seine, France) was immobilized at three densities on the flow cells of a CMS biosensor chip. Investigational FVIII samples were diluted to a series of five dilutions (0.18 to 5 nM FVIII according to the given protein values) with running buffer (10 mM Hepes, 150 mM NaCl, 0.05% Surfactant P20, pH 7.4), then applied to the chip using “single cycle” mode with a constant flow rate of 50 μL/min. Time for association was 4 min and that for dissociation was 10 min. After each cycle, FVIII was removed from the chip (“regeneration”) and the experiment repeated with a new FVIII sample. Association and dissociation constants were determined using the Langmuir model of the ‘Bioevaluation’ program. The following kinetic parameters were determined: Association rate constant ka, dissociation rate constant kd and equilibrium dissociation constant KD. Binding was also determined by evaluating Rmax, the calculated maximum binding at saturation. The kinetic results were calculated from the mean of the three different VWF immobilization levels.

Biacore technology was used to determine the kinetics of the complex formation between VWF and FVIII. For this purpose, plasma-derived VWF was immobilized onto three different levels on the sensor chip surface and the investigational PSA-rFVIII and rFVIII rebuffered into the buffer described in Example 13, above (“rebuffered FVIII”). Samples injected at five different concentrations in a single cycle mode. Association and dissociation constants were determined, assuming a homogeneous 1:1 interaction between the immobilized VWF and FVIII, using the Langmuir model of the “Bioevaluation” program of the Biacore T200 equipment.

Table 1 summarizes the kinetic parameters describing the VWF-FVIII interaction, where ka is the association rate constant, kd the dissociation rate constant and KD the equilibrium dissociation constant (=kd/ka). For further evaluation and data comparison, the mean and 3 SD of the various sample groups was calculated for KD.

TABLE 1
Kinetic parameters of VWF-FVIII binding

| Description | | Batch/lot Number | kₐ (1/Ms) | k_{d} (1/s) | K_{D} (nM) |
|---|---|---|---|---|---|
| PSA-rFVIII BDS | Preclinic | LOT A | 4.42E+06 | 8.49E−04 | 0.26 |
| | | LOT B | 3.10E+06 | 8.46E−04 | 0.33 |
| | | LOT C | 3.42E+06 | 6.76E−04 | 0.24 |
| | | mean | | | 0.28 |
| | | 3 SD | | | 0.15 |
| | GMP clinical | LOT D | 2.11E+06 | 5.74E−04 | 0.28 |
| | phase 1 | LOT E | 3.06E+06 | 7.24E−04 | 0.24 |
| | | LOT F | 2.91E+06 | 9.25E−04 | 0.33 |
| | | mean | | | 0.28 |
| | | 3 SD | | | 0.15 |
| PSA-rFVIII FDP | Preclinic | LOT G | 2.93E+06 | 9.77E−04 | 0.39 |
| | | LOT H | 3.45E+06 | 7.33E−04 | 0.27 |
| | | LOT I | 2.73E+06 | 6.64E−04 | 0.26 |
| | | mean | | | 0.31 |
| | | 3 SD | | | 0.21 |
| | GMP clinical | LOT J | 2.97E+06 | 8.51E−04 | 0.37 |
| | phase 1 | | | | |
| Rebuffered rFVIII for | Preclinic | LOT K | 3.28E+06 | 7.22E−04 | 0.26 |
| PSA-rFVIII | | LOT L | 2.49E+06 | 6.34E−04 | 0.29 |
| | GMP clinical | LOT M | 2.25E+06 | 8.02E−04 | 0.37 |
| | phase 1 | LOT N | 2.28E+06 | 7.39E−04 | 0.34 |

| | |
|---|---|
| mean | 0.32 |
| 3 SD | 0.15 |

Interaction kinetics were similar between the preclinical BDS batches (mean KD 0.28 nM) and the clinical phase 1 BDS batches (mean KD 0.28 nM), and between the preclinical FDP batches (mean KD 0.31 nM) and the clinical phase 1 FDP lot (KD 0.37 nM). For rebuffered rFVIII, KD values ranged from 0.26 and 0.37 nM and were thus comparable to PSA-rFVIII.

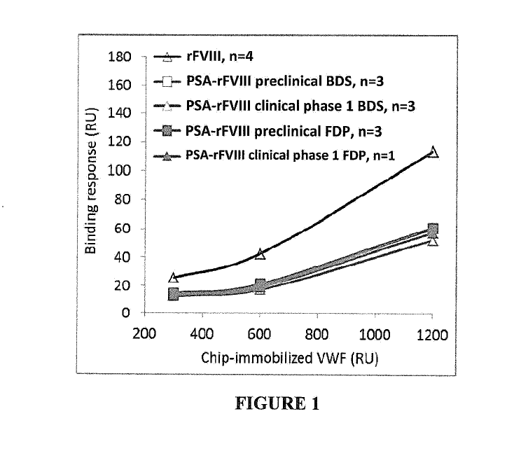

FIG. 1 shows binding signals expressed as Rmax, which is the calculated maximum binding at saturation, for PSA-rFVIII groups and rebuffered rFVIII at the three different densities of the sensor-chip-immobilized VWF. Both PSA-rFVIII and rebuffered rFVIII showed VWF concentration-dependent interaction with no relevant differences between PSA-rFVIII preclinical and clinical phase BDS and FDP batches. Compared with rebuffered rFVIII, the binding of PSA-rFVIII was markedly reduced by approximately 50%. This was considered to be a result of PSA modification of rFVIII, which yields a rFVIII conjugate where specific binding epitopes for VWF are shielded by PSA.

Interestingly and unlike the above binding properties observed with PSA-rFVIII compared to rebuffered rFVIII, PEG-rFVIII (a PEGylated rFVIII protein) also showed reduced binding to VWF but was not as pronounced as PSA-rFVIII.

### Example 15

### Determination of the FVIII-LRP1 Receptor Interaction by Surface Plasmon Resonance (SPR)

LRP1 (α2-macroglobulin receptor/CD91) receptor (BioMac, Leipzig, Germany) was immobilized on the flow cells of a CM4 sensor chip of a Biacore instrument (GE Healthcare, Uppsala, Sweden) to a constant level according to the manufacturer's instructions. A series of dilutions (21 to 357 nM, according to the given protein values) of investigated FVIII samples were then applied to the chip using the “kinject” mode, allowing 10 min for association and 5 min for dissociation of FVIII. After each cycle, FVIII was removed from the chip (“regeneration”) and the experiment repeated with a new FVIII sample. Association and dissociation constants were determined using the Langmuir model of the ‘Bioevaluation’ program, assuming a homogeneous 1:1 interaction. The following kinetic parameters were determined: Association rate constant ka, dissociation rate constant kd and equilibrium dissociation constant KD. Binding was also evaluated by determining the signal (response units) after the association phase. The kinetic results were calculated from the mean of the three different flow cells.

Binding affinity of PSA-rFVIII to the clearance receptor LRP1 was determined using Biacore technology as described above. Binding was tested in a dilution series of samples, diluted from 10 to 100 μg/mL (corresponding to 21 to 357 nM). Analysis was thus possible for the PSA-rFVIII BDS batches and rebuffered rFVIII, but not for the FDP lots due to the low protein concentration of these samples. The following parameters were determined: ka is the association rate constant, kd the dissociation rate constant and KD the equilibrium dissociation constant (=kd/ka). For further evaluation and data comparison, the mean and 3 SD of the various sample groups was calculated for KD.

Table 2 summarizes the kinetic parameters of the interaction between PSA-rFVIII preclinical and clinical phase 1 BDS and re-buffered rFVIII with LRP1 receptor. Interaction kinetics were similar between the preclinical (mean KD) and clinical phase 1 BDS batches (mean KD). Moreover, the binding kinetics were similar between PSA-rFVIII and re-buffered rFVIII. Due to the higher variability of surface plasmon resonance assays in general and evaluation of kinetic binding parameters, the variation in KD between the several samples is not regarded as biologically relevant and in summary confirmed that PSA-rFVIII preclinical and clinical phase 1 batches have similar properties.

TABLE 2
Kinetic parameters of LRP1-FVIII binding

| Description | | Batch/lot Number | kₐ (1/Ms) | k_{d} (1/s) | K_{D} (nM) |
|---|---|---|---|---|---|
| PSA-rFVIII BDS | Preclinic | LOT A | 2.45E+04 | 3.09E−04 | 12.6 |
| | | LOT B | 4.70E+03 | 1.24E−04 | 26.4 |
| | | LOT C | 2.08E+04 | 3.76E−04 | 18.1 |
| | | mean | | | 19.0 |
| | | 3 SD | | | 20.7 |
| | GMP clinical | LOT D | 4.57E+03 | 1.32E−04 | 28.9 |
| | phase 1 | LOT E | 1.27E+04 | 3.32E−04 | 26.0 |
| | | LOT F | 4.53E+03 | 1.86E−04 | 41.0 |
| | | mean | | | 32.0 |
| | | 3 SD | | | 24.0 |
| Rebuffered rFVIII for | Preclinic | LOT K | 1.70E+04 | 4.76E−04 | 28.0 |
| PSA-rFVIII | | LOT L | 3.73E+04 | 8.80E−04 | 23.6 |
| | GMP clinical | LOT M | 2.09E+04 | 3.95E−04 | 18.9 |
| | phase 1 | LOT N | 2.92E+04 | 5.43E−04 | 18.6 |

| | |
|---|---|
| mean | 22.3 |
| 3 SD | 13.5 |

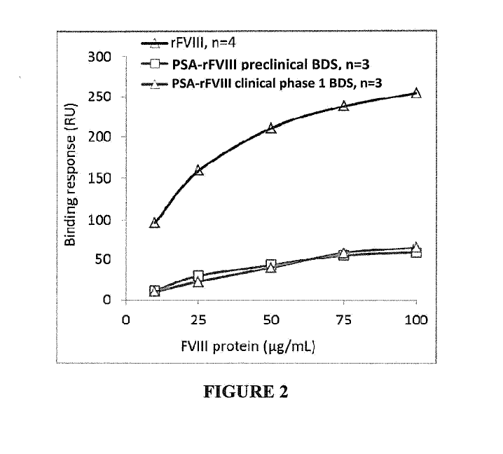

FIG. 2 shows results for FVIII protein-concentration-dependent binding to LRP1 for PSA-rFVIII and for re-buffered rFVIII. FVIII protein concentrations were plotted against the binding signal, expressed as response units. Both PSA-rFVIII and rebuffered rFVIII showed a FVIII concentration-dependent interaction with no relevant differences between the preclinical and clinical phase 1 BDS batches of PSA-rFVIII. Compared to rebuffered rFVIII, the binding of PSA-rFVIII was markedly reduced. This was considered to be a result of PSA modification of rFVIII that yields a rFVIII conjugate where specific binding epitopes for LRP1 are shielded by PSA.

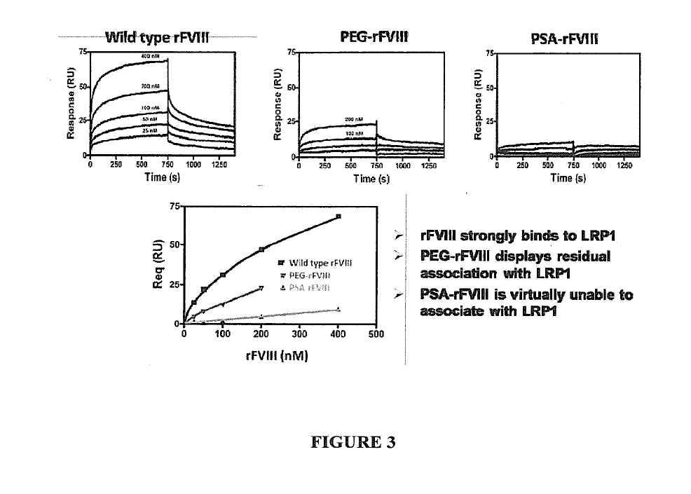

Interestingly, comparing the binding activity of rFVIII, PEG-rFVIII and PSA-rFVIII shows that rFVIII binds strongly to LRP1, PEG-rFVIII displayed residual association with LRP1, and PEG-rFVIII was virtually unable to associate with LRP1 (FIG. 3)

### Example 16

### Determination of FIXa-Cofactor Activity

The FIXa-cofactor activity of FVIII within the tenase-complex of PSA-rFVIII was assessed in vitro using a FIXa-cofactor activity assay. This assay provides detailed insight into kinetic properties of FXa generation. The samples were diluted to 1.0 IU/mL of FVIII activity according to their given potencies and time course of FXa generation after thrombin activation was measured.

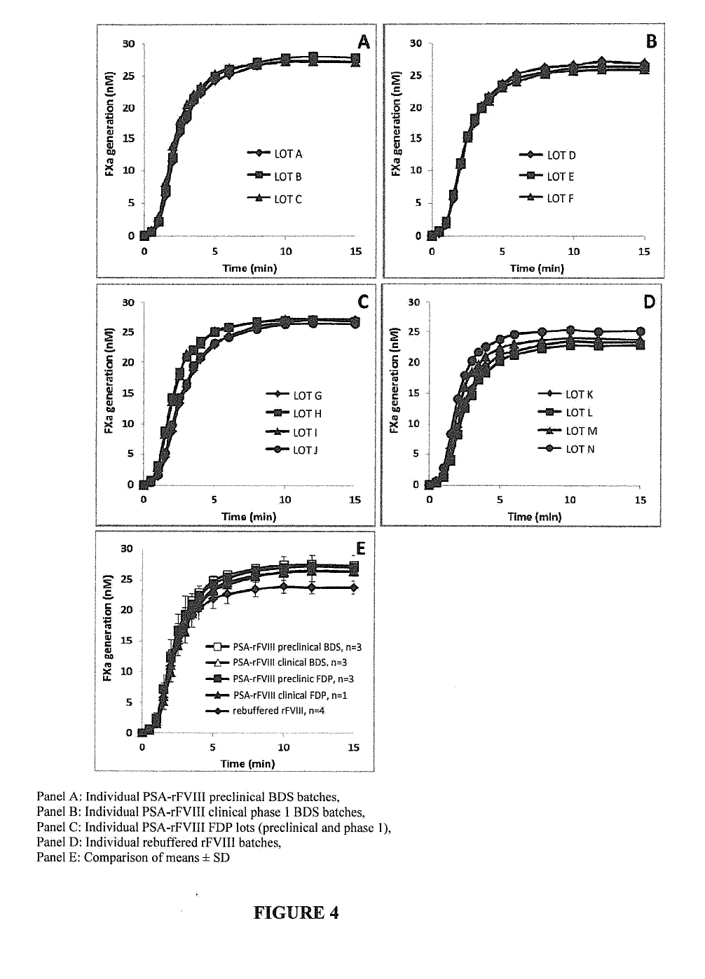

FIG. 4 shows FXa generation over time after activation of FVIII by thrombin. All PSA-rFVIII and rebuffered rFVIII batches showed time-dependent FXa generation, with only minor differences between the batches (FIG. 4, panel A-D). Comparison of the group means (FIG. 4, panel E) confirmed that PSA-rFVIII preclinical and clinical phase 1 BDS and FDP batches have similar properties. Some differences in the FXa generation curves were observed between rebuffered rFVIII and PSA-rFVIII (FIG. 4, panel E), e.g. rebuffered rFVIII showed slightly lower maximum FXa generation.

To evaluate comparability between the various sample groups on a quantitative basis, the maximum rate of FX activation was calculated by determining the slope of the linear part of the curve. Results are shown in Table 3. For direct comparison relative differences between the individual batches and the arithmetic means of the preclinical BDS or FDP batches (Table 4). The relative difference between the individual clinical phase 1 BDS batches and the mean of preclinical BDS batches was ≦11%, which confirms the comparability of the two sample groups. The differences between the clinical phase 1 PSA-rFVIII FDP and the mean of preclinical FDP were ≦11%, again demonstrating their similar characteristics. A relative comparison between the various PSA-rFVIII groups and the mean of rebuffered rFVIII is shown in Table 5. The differences were ≦6%, thus demonstrating that PSA-rFVIII and rebuffered rFVIII have similar kinetic properties of FXa generation.

TABLE 3
FXa generation parameters after thrombin pre-activation
of FVIII

| | | | Maximum rate |
|---|---|---|---|
| Usage | | Batch/lot Number | (nM/min) |
| PSA-rFVIII BDS | Preclinic | LOT A | 9.58 |
| | | LOT B | 10.10 |
| | | LOT C | 10.92 |
| | | mean | 10.20 |
| | | 3 SD | 2.04 |
| | GMP | LOT D | 9.52 |
| | clinical | LOT E | 9.14 |
| | phase 1 | LOT F | 9.05 |
| | | mean | 9.24 |
| | | 3 SD | 0.75 |
| PSA-rFVIII FDP | Preclinic | LOT G | 8.95 |
| | | LOT H | 11.15 |
| | | LOT I | 10.90 |
| | | mean | 10.33 |
| | | 3 SD | 3.60 |
| | GMP | LOT J | 9.19 |
| | clinical | | |
| | phase 1 | | |
| Rebuffered rFVIII for | Preclinic | LOT K | 9.01 |
| PSA-rFVIII | | LOT L | 8.67 |
| | GMP | LOT M | 9.88 |
| | clinical | LOT N | 11.35 |
| | phase 1 | | |
| | | mean | 9.73 |
| | | 3 SD | 3.60 |

TABLE 4
FXa generation parameters: Relative differences to mean of
preclinical PSA-rFVIII BDS and FDP batches

| | | |
|---|---|---|
| | | Maximum |
| Usage | Batch/lot Number | rate (%) |

| | | | |
|---|---|---|---|
| PSA- | Preclinic | mean | =100 |
| rFVIII BDS | GMP clinical | LOT D | 93 |
| | phase 1 | LOT E | 90 |
| | | LOT F | 89 |
| PSA- | Preclinic | mean | =100 |
| rFVIII FDP | GMP clinical | LOT J | 89 |
| | phase 1 | | |

TABLE 5
FXa generation parameters: Relative differences between PSA-
rFVIII groups and rebuffered rFVIII

| | maximum rate |
|---|---|
| Usage | (%) |
| Rebuffered rFVIII (mean, n = 4) | =100 |
| PSA-rFVIII BDS preclinic (mean, n = 3) | 105 |
| PSA-rFVIII FDP preclinic (mean, n = 3) | 106 |
| PSA-rFVIII BDS GMP clinic phase 1 (mean, n = 3) | 95 |
| PSA-rFVIII FDP GMP clinic phase 1 (n = 1) | 94 |

### Example 17

### Thrombin Generation Assay (TGA)

TGA is a specific method to study thrombin generation in an environment resembling the situation in hemophilia A patients. Human FVIII-deficient plasma containing <1% of FVIII was supplemented with increasing amounts of PSA-rFVIII (0.01 to 1 IU/mL, based on their given potencies). The reaction was started by adding a small amount of recombinant human tissue factor complexed with phospholipid micelles to the plasma to simulate a small vessel wall injury.

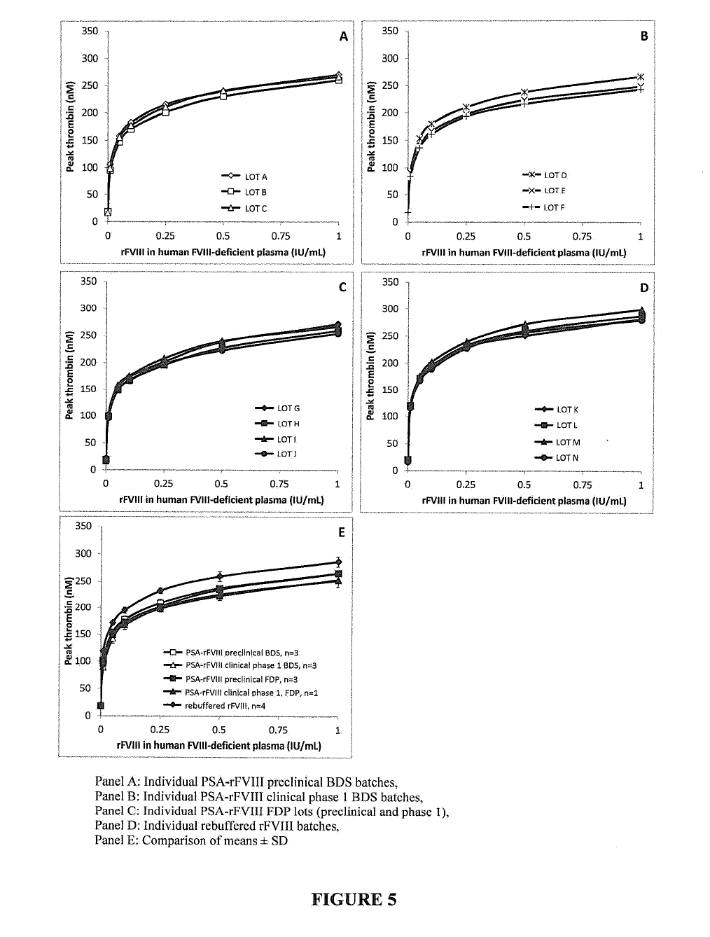

Thrombin generation was evaluated by comparing the concentration-dependent increase of the peak thrombin of the curves (FIG. 5). Both PSA-rFVIII and rebuffered rFVIII showed a FVIII-concentration-dependent increase in peak thrombin with only minimal variations between the individual batches (FIG. 5, panels A to D). Comparison of the group means (FIG. 5, panel E) confirmed that PSA-rFVIII preclinical and clinical phase 1 BDS and FDP batches had similar properties. Differences in peak thrombin generation were observed between rebuffered rFVIII and PSA-rFVIII (FIG. 5, panel E).

For a more thorough evaluation, a quantitative comparison was made. Table 6 summarizes peak thrombin values measured at the different FVIII concentrations tested. The samples were comparatively analyzed by calculating the area under the curve (AUC) of peak thrombin for each one. Moreover, relative differences in the AUC of peak thrombin values of the individual PSA-rFVIII clinical phase 1 BDS/FDP batches and the arithmetic means of the preclinical BDS/FDP batches were calculated.

The relative differences between values for the individual clinical phase 1 BDS batches and the mean for preclinical BDS batches were <9%, which confirm comparability between the two sample groups. For clinical phase 1 FDP, the difference to the mean for preclinical FDP was ≦4%, again showing highly similar characteristics.

Evaluation of the peak thrombin generation curves (FIG. 5) showed slightly higher thrombin generation for rebuffered rFVIII than for PSA-rFVIII. Since the relative differences in the calculated AUC of the several PSA-rFVIII groups and the mean for rebuffered rFVIII were ≦14%, this result was regarded to be of minor relevance and more related to the inherent assay variation than to actual physiological relevant differences.

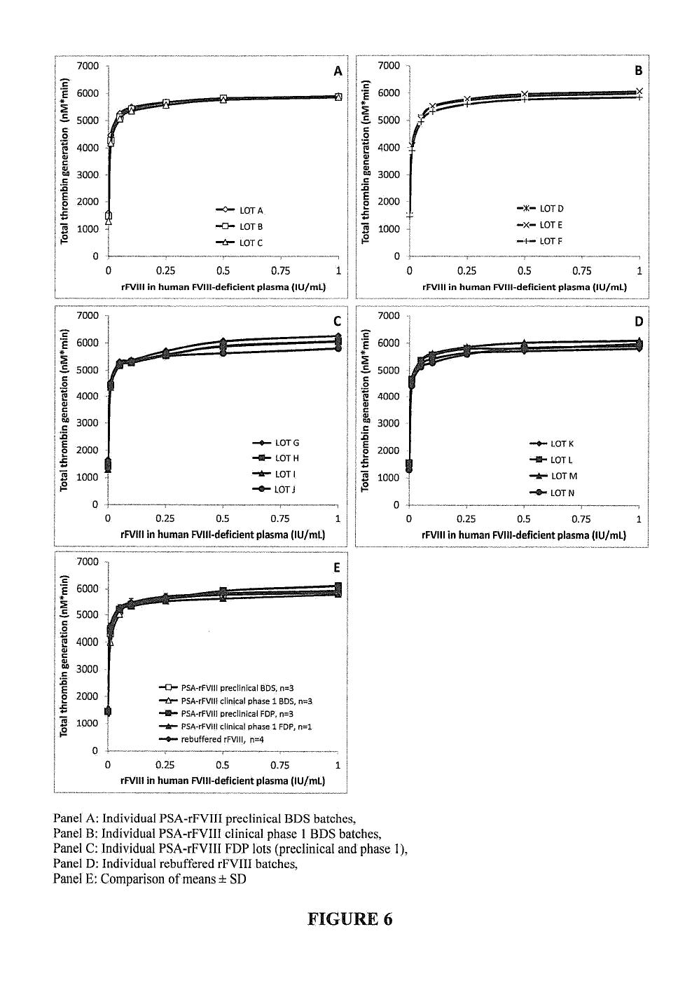

Thrombin generation was further evaluated by determining total thrombin generation. All samples of PSA-rFVIII and rebuffered rFVIII BDS investigated showed a similar FVIII-concentration dependent increase in total thrombin generation, with minimal differences between the groups (FIG. 6). Compared with peak thrombin generation, the differences between PSA-rFVIII and rebuffered rFVIII were even lower.

TABLE 6
Concentration-dependent peak thrombin increasing capacity of FVIII batches
Peak thrombin (nM)

| | | | FVIII (IU/mL) | | | | | | |
|---|---|---|---|---|---|---|---|---|---|
| | | | 0 | 0.01 | 0.05 | 0.1 | 0.25 | 0.5 | 1 |
| PSA-rFVIII BDS | Preclinic | LOT A | 19.7 | 105.4 | 157.5 | 182.6 | 215.4 | 240.0 | 270.7 |
| | | LOT B | 18.6 | 95.4 | 147.4 | 170.2 | 201.0 | 230.6 | 260.0 |
| | | LOT C | 16.7 | 99.0 | 155.4 | 177.1 | 210.9 | 241.2 | 266.4 |
| | | mean | 18.3 | 99.9 | 153.4 | 176.6 | 209.1 | 237.3 | 265.7 |
| | | 3 SD | 4.5 | 15.3 | 15.9 | 18.6 | 22.2 | 17.4 | 16.2 |
| | GMP | LOT D | 18.3 | 93.8 | 152.9 | 179.8 | 210.3 | 238.0 | 266.4 |
| | clinical | LOT E | 18.6 | 91.3 | 137.1 | 166.2 | 196.6 | 223.3 | 248.2 |
| | phase 1 | LOT F | 18.0 | 83.8 | 136.4 | 160.7 | 193.3 | 216.0 | 243.0 |
| | | mean | 18.3 | 89.6 | 142.1 | 168.9 | 200.1 | 225.8 | 252.5 |
| | | 3 SD | 0.9 | 15.6 | 27.9 | 29.4 | 27.0 | 33.6 | 36.9 |
| PSA-rFVIII FDP | Preclinic | LOT G | 20.4 | 102.6 | 154.0 | 173.7 | 201.7 | 238.6 | 270.8 |
| | | LOT H | 17.9 | 98.9 | 148.7 | 167.0 | 195.8 | 227.5 | 259.3 |
| | | LOT I | 16.6 | 103.7 | 158.0 | 175.6 | 208.3 | 239.8 | 267.0 |
| | | mean | 18.3 | 101.7 | 153.6 | 172.1 | 201.9 | 235.3 | 265.7 |
| | | 3 SD | 5.7 | 7.5 | 14.1 | 13.5 | 18.9 | 20.4 | 17.7 |
| | GMP | LOT J | 19.3 | 96.4 | 149.1 | 167.6 | 197.9 | 223.2 | 253.8 |
| | clinical | | | | | | | | |
| | phase 1 | | | | | | | | |
| Rebuffered rFVIII for | Preclinic | LOT K | 18.0 | 119.7 | 169.7 | 191.6 | 230.3 | 250.8 | 281.6 |
| PSA-rFVIII | | LOT L | 19.6 | 119.5 | 172.0 | 195.6 | 232.7 | 259.6 | 287.6 |
| | GMP | LOT M | 19.6 | 121.4 | 175.7 | 202.4 | 239.5 | 271.8 | 299.2 |
| | clinical | LOT N | 16.3 | 116.3 | 167.7 | 189.3 | 228.0 | 256.7 | 279.0 |
| | phase 1 | | | | | | | | |
| | | mean | 18.4 | 119.2 | 171.3 | 194.7 | 232.6 | 259.7 | 286.9 |
| | | 3 SD | 4.8 | 6.3 | 10.2 | 17.1 | 15.0 | 26.4 | 27.0 |

TABLE 7
Peak thrombin generation capacity - Relative differences
to mean preclinical PSA-rFVIII BDS and FDP batches

| | | Batch/lot | AUC | AUC |
|---|---|---|---|---|
| Usage | | Number | (nM * IU/mL) | (%) |
| PSA-rFVIII BDS | Preclinic | LOT A | 228.8 | |
| | | LOT B | 217.7 | |
| | | LOT C | 226.5 | |
| | | mean | 224.3 | =100 |
| | | 3SD | 17.7 | |
| | GMP clinical | LOT D | 225.2 | 100 |
| | phase 1 | LOT E | 210.3 | 94 |
| | | LOT F | 204.8 | 91 |
| | | mean | 213.4 | 95 |
| | | 3SD | 31.8 | |
| PSA-rFVIII FDP | Preclinic | LOT G | 224.5 | |
| | | LOT H | 215.2 | |
| | | LOT I | 225.8 | |
| | | mean | 221.8 | =100 |
| | | 3SD | 17.4 | |
| | GMP clinical | LOT J | 212.7 | 96 |
| | phase 1 | | | |
| Rebuffered rFVIII | Preclinic | LOT K | 240.5 | |
| for PSA-rFVIII | | LOT L | 246.2 | |
| | GMP clinical | LOT M | 256.0 | |
| | phase 1 | LOT N | 241.1 | |
| | | mean | 246.0 | |
| | | 3SD | 21.6 | |

TABLE 8
Peak thrombin generation capacity - Relative differences
between PSA-rFVIII groups and rebuffered rFVIII

| | AUC | AUC |
|---|---|---|
| Usage | (nM * IU/mL) | (%) |
| Rebuffered rFVIII (mean, n = 4) | 246.0 | =100 |
| PSA-rFVIII BDS preclinic (mean, n = 3) | 224.3 | 91 |
| PSA-rFVIII FDP preclinic (mean, n = 3) | 221.8 | 90 |
| PSA-rFVIII BDS GMP clinic phase 1 (mean, | 213.4 | 87 |
| n = 3) | | |
| PSA-rFVIII FDP GMP clinic phase 1 (n = 1) | 212.7 | 86 |

### Example 18

Comparison of Pharmacokinetics of PSA-rFVIII, PEG-rFVIII and rFVIII in Hemophilic Mice

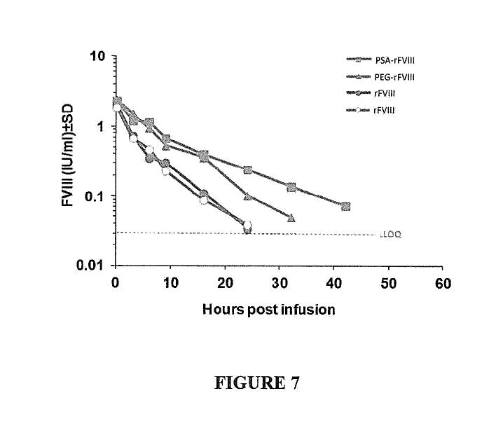

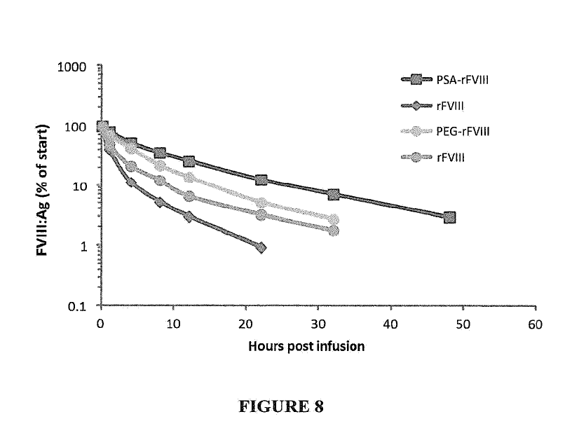

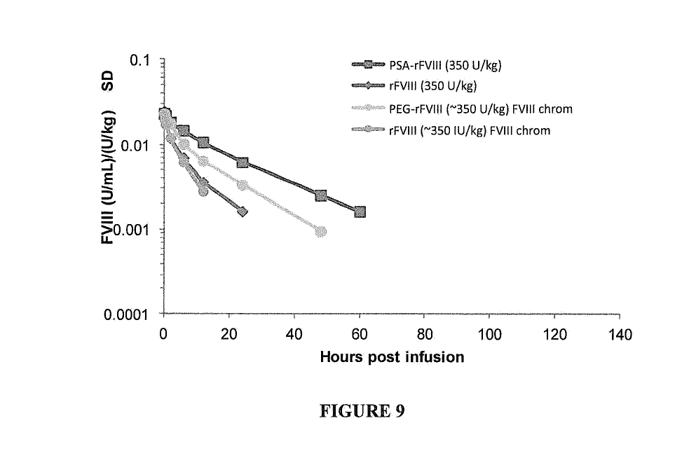

Pharmacokinetics of PSA-rFVIII, PEG-rFVIII and rFVIII were measured in FVIII knockout mice. Injection of 200 IU FVIII/kg bodyweight was administered via the tail vein. Groups of 6 mice were sacrificed after defined time points and citrated plasma is prepared. FVIII activity in plasma is measured with a chromogenic activity assay. As shown in FIG. 7 and Table 9, PEGylated and polysialylated rFVIII showed improved PK parameters compared to rFVIII (HL increase of ˜2). Similar results were observed in rats (FIG. 8) and in macaques (FIG. 9). (To allow comparison between different studies, data were normalized to a common dose of 1 U/kg).

TABLE 9

| | AUC | | | | | |
|---|---|---|---|---|---|---|
| | Dose | | Term. | | | |
| Lot | adjusted | Factor | HL h | Factor | MRT h | Factor |
| PSA- | 0.101 | 2.5 | 11.7 | 2.1 | 13.1 | 2.2 |
| rFVIII | | | | | | |
| PEG- | 0.085 | 1.6 | 6.3 | 1.0 | 7.8 | 1.5 |
| rFVIII | | | | | | |
| rFVIII | 0.041-0.053 | =1 | 5.5-6.0 | =1 | 6.1-5.2 | =1 |

### Example 19

### PSA-rFVIII In Vivo Efficacy

Efficacy of PSA-rFVIII was measured in a tail-clip bleeding model in the FVIII KO mouse. Application of 200 IU FVIII activity/kg was followed by transection of the tail tip at 18-54 hours after injection of PSA-rFVIII. Blood loss was measured, and the data confirmed longer efficacy for PSA-rFVIII as expected from PK studies. PSA-rFVIII controls bleeding approximately twice as long as rFVIII.

Efficacy was also measured in a carotid occlusion model in the FVIII KO mouse. Application of 200 IU FVIII activity/kg was followed by lesion of the carotis with ferrichloride. Time to vessel occlusion was measured and, consistent with the above results, data confirmed longer efficacy for PSA-rFVIII as expected from PK studies. An approximate 2 fold longer survival of PSA-rFVIII than rFVIII in FVIII KO mice was observed.

Finally, the effect of PEG-rFVIIII and PSA-rFVIII in a murine model of hemophilic joint bleeding (Intra-Articular Puncture) was assessed. Treatment with a modified rFVIII (i.e. PEG-rFVIII and PSA-rFVIII) was shown to last at least twice as long as for rFVIII in the murine model.

1. A modified Factor VIII (FVIII) comprising a modification that increases FVIII half-life and reduces binding of said modified FVIII to a ligand selected from the group consisting of von Willebrand Factor (VWF) and low density lipoprotein (LDL)-receptor-related protein 1 (LRP1).

2. The modified FVIII according to claim 1 wherein said modified FVIII is plasma-derived.

3. The modified FVIII according to claim 1 wherein said modified FVIII is recombinant.

4. The modified FVIII according to claim 3 wherein said modified FVIII is a full-length FVIII and includes an intact B domain.

5. The modified FVIII according to claim 1 wherein said FVIII binds to VWF and LRP1 with a lower affinity (KD) compared to unmodified FVIII.

6. The modified FVIII according to claim 1 wherein said modification comprises a polysialic acid (PSA).

7. The modified FVIII according to claim 6 wherein said PSA has a mean molecular size selected from the group consisting of approximately 20 kDa.

8. The modified FVIII according to claim 6 wherein said PSA has a low polydispersity.

9. The modified FVIII according to claim 6 wherein said PSA comprises an aminooxy linker.

10. The modified FVIII according to claim 9 wherein said aminooxy linker is attached to an oxidized carbohydrate of said modified FVIII.

11. A pharmaceutical composition comprising the modified FVIII according to claim 1 and a pharmaceutically acceptable carrier, diluent, salt, buffer, or excipient.

12. The modified FVIII of claim 6 wherein said half-life is longer that a PEGylated FVIII.

13. The modified FVIII of claim 12 wherein said half-life is longer by a factor of approximately 1, 2 or 3-fold.

14. The modified FVIII of claim 6 wherein said binding to VWF or LRP1 is lower as compared to binding of a PEGylated FVIII to VWF or LRP1.

15. The modified FVIII of claim 11 wherein said binding to VWF or LRP1 is lower by a factor of 0.5 as compared to binding of a PEGylated FVIII to VWF or LRP1.

16. A modified, recombinant FVIII comprising a modification that increases FVIII half-life and reduces binding of said modified FVIII to a ligand selected from the group consisting VWF and LRP1, wherein said modification comprises a PSA with an aminooxy linker, and wherein said aminooxy linker is attached to an oxidized carbohydrate of said modified FVIII;
wherein the half-life of said modified, recombinant FVIII is longer than an unmodified, recombinant FVIII and/or a PEGylated, recombinant FVIII; and
wherein the binding to VWF or LRP1 by said modified, recombinant FVIII is lower as compared to binding of VWF or LRP1 by an unmodified, recombinant FVIII and/or a PEGylated, recombinant FVIII.

17. The modified FVIII of any one of claims 1 and 16, wherein said modified FVIII is administered to a mammal diagnosed with disease or disorder associated with FVIII deficiency.

18. A method of treating a hemorrhagic defect in a mammal comprising the step of administering the modified FVIII of any one of claims 1 and 16, or the pharmaceutical composition of claim 11, to the mammal in an amount effective to reduce or eliminate one or more symptoms of said hemorrhagic defect.
